SEQUENCE LISTING (9 sequences)

SEQ ID NO: 1 - DNA, 933 bp, Neisseria meningitidis
  LgtC gene of Neisseria meningitidis (GenBank U65788)
atggacatcgtatttgcggcagacgacaactatgccgcctatctttgcgttgcggcaaaaagcgtggaagcggcccatcccgatacggaaatcaggttccacgtcctcgatgccggcatcagtgaggcaaaccgggcggcggttgctgccaatttgcggggggggggtaatatccgctttatagacgtaaaccccgaagatttcgccggcttccccttaaacatcaggcacatttccatcacgacttatgcccgcttgaaattgggcgaatacattgccgattgcgataaagtcctgtatctggatatagacgtattggtcagggacagcctgaagcccttatgggataccgatttgggcgataactggcttggcgcgtgcatcgatttgtttgtcgaaaggcagaatgcttacaaacaaaaaatcggtatggcggacggagaatattatttcaatgccggcgtattgctgatcaacctgaaaaagtggcggcagcacgatattttcaaaatggcctgcgaatgggtggaacaatacaaggacgtgatgcaatatcaggatcaggatattttgaacgggctgtttaaaggcggggtgtgttatgcgaacagccgtttcaactttatgccgaccaatgatgcctttatggcgaacaggtttgcgtcccgccataccgacccgctttaccgcgaccggacttatacggcgatgcctgtcgccgtcagccattattgcggcccggcaaagccgtggcacagggactgcaccgcgtggggtgcggaacgtttcacagaattggcgggcagcctgacgagcgttcccgaagaatggcgcggcaaacttgccgtcccgcaccgtgtgtttccgacaaagcgtatgcttcaaagatggcgcagaaagctgtctgccagattcttacgcaagatttattga

SEQ ID NO: 2 - protein, 323 aa, Neisseria meningitidis
  LgtC protein of Neisseria meningitidis (id AAB48385)
MENCPLVSVIVCAYNAEQYIDESISSIINQTYENLEIIVINDGSTDLTLSHLEEISKLDKRIKIISNKYNLGFINSLNIGLGCFSGKYFARMDADDIAKPSWIEKIVTYLEKNDHITAMGSYLEIIVEKECGIIGSQYKTGDIWKNPLLHNDICEAMLFYNPIHNNTMIMRANVYREHKLIFNKDYPYAEDYKFWSEVSRLGCLANYPEALVKYRLHGNQTSSVYNHEQNETAKKIKRENITYYLNKIGIDIKVINSVSLLEIYHVDKSNKVLKSILYEMYMSLDKYTITSLLHFIKYHLELFDLKQNLKIIKKFIRKINVIF

SEQ ID NO: 3 - DNA, 972 bp, Haemophilus influenzae
  LgtD gene from Haemophilus influenzae (GenBank U32832)
atggaaaattgtccattagtatcggttattgtttgtgcttataacgctgagcaatatatagatgaaagcatttcatccattattaatcagacttatgaaaatctagaaattatagttatcaatgatggttcaacagatttgactttgtctcatttagaagaaatatctaaattagataaaaggataaaaattatcagtaataaatataatttagggttcataaattctttgaatataggccttggttgtttttcaggtaaatattttgcaagaatggatgctgatgatatagctaaaccatcgtggattgagaaaatagttacctatctggagaaaaatgatcatattacagcaatgggatcatacttagagattattgtagaaaaagaatgtggaattatcggttctcaatataaaactggagatatatggaaaaatccattgctacataatgatatttgtgaagctatgcttttctataatccgatacataacaacactatgattatgagagcaaatgtatatagagagcataaattaatctttaataaagattatccgtatgcagaagattataagttttggtcagaggttagtaggcttggttgtttagctaattatcctgaagcattagtaaaatatagactacatggaaaccaaacatcatcagtttataatcatgagcaaaatgagacagctaaaaagataaagagggaaaatattacatattaccttaataagataggtatagatataaaagtaattaatagtgtgtcgttgctagaaatatatcatgtggataaaagtaataaagtgttgaaaagtatactttatgagatgtatatgagcttagataaatatactataacttcactcttacattttattaaatatcatcttgaattatttgatttaaagcaaaatttaaagattataaaaaagttcataagaaaaataaatgttatattttag

SEQ ID NO: 4 - protein, 323 aa, Haemophilus influenzae; identical to SEQ ID NO: 2
  LgtD protein from Haemophilus influenzae (id AAC23227)

SEQ ID NO: 5 - DNA, 944 bp, Helicobacter pylori
  Helicobacter pylori gene futC (GenBank AF076779)
atggcttttaaagtggtgcaaatttgtggggggcttgggaatcaaatgtttcaatacgctttcgctaaaagtttgcaaaaacaccttaatacgcccgtgctattagacactacttcttttgattggagcaataggaaaatgcaattagagcttttccctattgatttgccctatgcgaatgcaaaagaaatcgctatagctaaaatgcaacatctccccaagttagtaagagatgcactcaaatacataggatttgatagggtgagtcaagaaatcgtttttgaatacgagcctaaattgttaaagccaagccgtttgacttatttttttggctatttccaagatccacgatattttgatgctatatcctctttaatcaagcaaaccttcactctaccccccccccccgaaaataataaaaataataataaaaaagaggaagaataccagcgcaagctttctttgattttagccgctaaaaacagcgtatttgtgcatataagaagaggggattatgtggggattggctgtcagcttggtattgattatcaaaaaaaggcgcttgagtatatggcaaagcgcgtgccaaacatggagctttttgtgttttgcgaagacttaaaattcacgcaaaatcttgatcttggctaccctttcacggacatgaccactagggataaagaagaagaggcgtattgggatatgctgctcatgcaatcttgcaagcatggcattatcgctaatagcacttatagctggtgggcggcttatttgatggaaaatccagaaaaaatcattattggccccaaacactggctttttgggcatgaaaatattctttgtaaggaatgggtgaaaatagaatcccattttgaggtaaaatcccaaaaatataacgcttaaagcggcttaaaaaaagggcttactagaggtttaatctttga

SEQ ID NO: 6 - protein, 300 aa, Helicobacter pylori
  Helicobacter pylori futC protein id (id AAC99764)
MAFKVVQICGGLGNQMFQYAFAKSLQKHLNTPVLLDTTSFDWSNRKMQLELFPIDLPYANAKEIAIAKMQHLPKLVRDALKYIGFDRVSQEIVFEYEPKLLKPSRLTYFFGYFQDPRYFDAISSLIKQTFTLPPPPENNKNNNKKEEEYQRKLSLILAAKNSVFVHIRRGDYVGIGCQLGIDYQKKALEYMAKRVPNMELFVFCEDLKFTQNLDLGYPFTDMTTRDKEEEAYWDMLLMQSCKHGIIANSTYSWWAAYLMENPEKIIIGPKHWLFGHENILCKEWVKIESHFEVKSQKYNA

SEQ ID NO: 7 - DNA, 1116 bp, Neisseria meningitidis
  Alpha-2,3-sialyltransferase of N. meningitidis (GenBank U60660)
atgggcttgaaaaaggcttgtttgaccgtgttgtgtttgattgttttttgtttcgggatattttatacatttgaccgggtaaatcagggggaaaggaatgcggtttccctgctgaaggagaaacttttcaatgaagagggggaaccggtcaatctgattttctgttataccatattgcagatgaaggtggcggaaaggattatggcgcagcatccgggcgagcggttttatgtggtgctgatgtctgaaaacaggaatgaaaaatacgattattatttcaatcagataaaggataaggcggagcgggcgtactttttccacctgccctacggtttgaacaaatcgtttaatttcattccgacgatggcggagctgaaggtaaagtcgatgctgctgccgaaagtcaagcggatttatttggcaagtttggaaaaagtcagcattgccgcctttttgagcacttacccggatgcggaaatcaaaacctttgacgacgggacaggcaatttaattcaaagcagcagctatttgggcgatgagttttctgtaaacgggacgatcaagcggaattttgcccggatgatgatcggagattggagcatcgccaaaacccgcaatgcttccgacgagcattacacgatattcaagggtttgaaaaacattatggacgacggccgccgcaagatgacttacctgccgctgttcgatgcgtccgaactgaagacgggggacgaaacgggcggcacggtgcggatacttttgggttcgcccgacaaagagatgaaggaaatttcggaaaaggcggcaaaaaacttcaaaatacaatatgtcgcgccgcatccccgccaaacctacgggctttccggcgtaaccacattaaattcgccctatgtcatcgaagactatattttgcgcgagattaagaaaaacccgcatacgaggtatgaaatttatacctttttcagcggcgcggcgttgacgatgaaggattttcccaatgtgcacgtttacgcattgaaaccggcttcccttccggaagattattggctcaagccggtgtatgccctgtttacccaatccggcatcccgattttgacatttgacgataaaaattaa

SEQ ID NO: 8 - protein, 371 aa, Neisseria meningitidis
  Alpha-2,3-sialyltransferase of N. meningitidis (id AAC44541.1)
MGLKKACLTVLCLIVFCFGIFYTFDRVNQGERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 9 - protein, 328 aa, Campylobacter jejuni
  UDP-glucose 4-epirase Campylobacter jejuni subsp. jejuni (NCTC 11168 - NCBI CAB73386.1)
MKILISGGAGYIGSHTLRQFLKTDHEICVLDNLSKGSKIAIEDLQKTRAFKFFEQDLSDFQGVKALFEREKFDAIVHFAASIEVFESMQNPLKYYMNNTVNTTNLIETCLQTGVNKFIFSSTAATYGEPQTPVVSETSPLAPINPYGRSKLMSEEVLRDASMANPEFKHCILRYFNVAGACMDYTLGQRYPKATLLIKVAAECAAGKRDKLFIFGDDYDTKDGTCIRDFIHVDDISSAHLAALDYLKENESNVFNVGYGHGFSVKEVIEAMKKVSGVDFKVELAPRRAGDPSVLISDASKIRNLTSWQPKYDDLELICKSAFDWEKQC

(19) United States
(12) Patent Application Publication
(10) Pub. No.: US 2007/0202578 A1
(43) Pub. Date: Aug. 30, 2007

(54) Production of globosides oligosaccharides using metabolically engineered microorganisms

(75) Inventors: Eric Samain, Gieres (FR); Mialy Randriantsoa, Saint Martin D'Heres (FR); Sophie Drouillard, Claix (FR)

Correspondence Address: FOLEY AND LARDNER LLP;SUITE 500, 3000 K STREET NW, WASHINGTON, DC, 20007, US

(73) Assignee: CENTRE NATIONAL DE LA RECHERCHE SCIENTIFIQUE (CNRS)

(21) Appl. No.: 11/509,818

(22) Filed: Aug. 25, 2006

Related U.S. Application Data
(60) Provisional application No. 60/711,406, filed on Aug. 26, 2005.

(51) Int. Cl. C12P 19/04; C12N 15/74; C12N 1/21

(52) U.S. Cl. 435/101; 435/252.3; 435/252.33; 435/471

(57) ABSTRACT

The present invention relates to the large scale in vivo synthesis of globosides oligosaccharides; especially globotriose, globotetraose and globopentaose, which are the carbohydrate portions of globotriosylceramide (Gb3Cer), globotetraosylceramide (Gb4Cer) and globopentaosylceramide (Gb5Cer) respectively. It also relates to high yield production of potential anticancer vaccines of the globo-series glycosphingolipids, including the Globo-H. It also relates to the use of the glycosyltransferase encoded by the lgtD gene from Haemophilus influenzae as a β1,3 galactosyl transferase to catalyze the transfer of a galactose moiety from UDP-Gal to an acceptor bearing the terminal non reducing structure GalNAcβ-3-R to form the Galβ-3GalNAcβ-3-R structure

### FIELD OF THE INVENTION

The present invention relates to the large scale in vivo synthesis of globosides oligosaccharides; especially globotriose, globotetraose and globopentaose, which are the carbohydrate portions of globotriosylceramide (Gb3Cer), globotetraosylceramide (Gb4Cer) and globopentaosylceramide (Gb5Cer) respectively. It also relates to high yield production of potential anticancer vaccines carbohydrate epitope of the globo-series glycosphingolipids, including Globo-H. It also relates to the use of the glycosyltransferase encoded by the lgtD gene from Haemophilus influenzae as a β1,3 galactosyl transferase to catalyze the transfer of a galactose moiety from UDP-Gal to an acceptor bearing the terminal non reducing structure GalNAcβ-3-R to form the Galβ-3GalNAcβ-3-R structure

### BACKGROUND OF THE INVENTION

It is now well-established that oligosaccharides play an important biological role especially as regards to the activity and function of proteins; thus, they serve to modulate the half-life of proteins, and occasionally they are involved in the structure of the protein. Oligosaccharides play an essential role in antigen variability (for example blood groups), and in certain bacterial infections such as those caused by Neisseria meningitidis.

As oligosaccharides are usually obtained in a low yield by purification starting from natural sources, the synthesis of oligosaccharides has become a major challenge of carbohydrate chemistry. In particular, it is a goal to supply sufficient amounts of well-characterized oligosaccharides, required for fundamental research or for any other potential applications.

The synthesis of complex oligosaccharides of biological interest may be performed chemically, enzymatically or microbiologically. Despite the development of new chemical methods for synthesizing oligosaccharides in the course of the last 20 years, the chemical synthesis of oligosaccharides remains very difficult on account of the numerous selective protection and deprotection steps, the lability of the glycoside linkages, the difficulties in obtaining regiospecific couplings, and the low production yields.

High yield production and purification of globosides has remained a challenge despite substantial utilities of these particular oligosaccharides. For example, Gb3Cer constitutes the rare P^{k} blood group antigen on erythrocytes and the CD77 differentiation antigen on lymphocytes. Gb3Cer is also the receptor for the Shiga toxins (Stx) produced by Shigella dysenteriae [1] and Stx-like producing Escherichia coli strains. Gb4Cer, known as globoside or P antigen, is the most abundant neutral glycosphingolipid in erythrocyte membranes. The P antigen has been identified as the receptor for the parvo-B19 virus [2], which was shown to bind erythroid progenitor cells via interaction with three globotetraose molecules [3]. The galabiose motif (Galα-4Gal) present in Gb3Cer, Gb4Cer and Gb5Cer is the minimal structure recognized by the PapG adhesins that are found at the tip of pili on uropathogenic Escherichia coli strains [4].

Globotriose has also been found in meningococcal lipooligosaccharides [5] and the lgtC gene encoding the α1,4 galactosyltransferase has been identified in both Neisseria gonorrhoeae[6] and N. meningitidis [7]. Recombinant LgtC enzyme has been successfully overproduced in E. coli [8] and used for large scale enzymatic synthesis of globotriose using purified enzymes [9], [10] or metabolically engineered permeabilized whole cells [11], [12]. Expression of a globotetraose epitope in a bacterial lipopolysaccharide was first reported in a capsule deficient strain of Haemophilus influenzae Rd [13]. The lgtD gene for β1,3-N-acetylgalactosaminyltransferase was later identified in H. influenzae Rd [14] and further characterized [10]. The enzymatic synthesis of globotetraose was reported using purified recombinant H. influenzae LgtD protein and an enzymatic UDP-GalNAc regeneration system ([15] [16]).

In addition, the globopentaose is often referred to as the stage specific embryonic antigen-3 (SSEA-3) which is the carbohydrate moiety of the galactosyl-globoside (Gb5). Gb5 is expressed by human embryonal carcinoma cells [27] and is a key intermediate for the synthesis of other tumor makers and potential anticancer vaccines of the globo-series glycosphingolipids, which include the Globo-H [28], the sialosyl galactosyl globoside [29] and the disialosyl galactosyl globoside [31]. The sialosyl galactosyl globoside was also found to be the preferred binding receptor for uropathogenic Escherichia coli [30] and could potentially be used as an anti-infective agent.

We have recently developed a new fermentation process for the low-cost production of lactose-derived oligosaccharides using living recombinant E. coli cells overexpressing the suitable glycosyltransferase genes [17]. The process is based on the active uptake of lactose while the cells are growing on an alternative substrate such as glycerol. Here, we go further and provide a new process which can be advantageously used for the large scale production of the above mentioned globosides oligosaccharides. This process is also based on the discovery the β-3 Gal transferase activity encoded by lgtD is capable of efficient convertion of globotetraose into globopentaose. In addition, we have designed and practiced a method which benefits from the observation that Escherichia coli cells are able to efficiently internalize exogenous globotriose into the cells.

### BRIEF SUMMARY OF THE INVENTION

The present invention provides methods of producing oligosaccharides by fermentative growth of microorganisms. In particular, the invention relates to a method of synthesis of “globosides” which will be understood as oligosaccharides comprising the galabiose motif (Galα-4Gal) including but not limited to:

(1) globotriose (Galα-4Galβ-4Glc),

(4) globotetraose (GalNAcβ-3Galα-4Galβ-4Glc),

(11) globopentaose (Galβ-3GalNAcβ-3Galα-4Galβ-4Gal),

(12) globo-H hexasaccharide (Fucα-2Galβ-3GalNAcβ-3Galα-4Galβ-4Gal),

(13) sialosyl galactosyl globoside (SGG) hexasaccharide (NeuAcα-3Galβ-3GalNAcβ-3Galα-4Galβ-4Gal), as well as

(14) disialosyl galactosyl globoside heptasaccharide (NeuAcα-3Galβ3-3(NeuAcα-6)GalNAβ-3Galα-4Galβ-4Gal)

using in a first common step an engineered micro-organism expressing, for example, the Neisseria lgtC gene for α-1,4-Gal transferase in specific culture conditions leading to production of globotriose without production of polygalactosylated side-products such as tetrasaccharide (Galα-4Galα-4Galβ-4Gal) and pentasaccharide (Galα-4Galα-4Galα-4Galβ-4Gal). In further steps, exogenous globotriose obtained above is supplied in the culture medium and is internalized by engineered micro-organisms to produce the other cited globosides sugars. This method may be extended to the production of Gb3Cer, Gb4Cer and Gb5Cer by reacting the above oligosaccharide moities with ceramide.

The present invention also provides methods of producing of “globosides” which will be understood as oligosaccharides comprising the motif Galβ-3GalNAcβ-3-R including but not limited to:

Galβ-3GalNAcβ-3Gal, Galβ-3GalNAcβ-3Galα-X, Galβ-3GalNAcβ-3Galβ-X, Galβ-3GalNAcβ-3Galβ-allyl, Galβ-3GalNAcβ-3Galo-propragyl, Galβ-3GalNAβ-3Galα-4Galβ-4Gal (Globopentaose), Galβ-3GalNAcβ-3Galα-4Galβ-4Galα-X, Galβ-3GalNAcβ-3Galα-4Galβ-4Galβ-X, Galβ-3GalNAcβ-3Galα-4Galβ-4Galβ-allyl, and Galβ-3GalNAcβ-3Galα-4Galβ-4Galβ-propargyl;

Using in a first common step an exogenous glycosyltransferase encoded by the lgtD gene from Haemophilus influenzae of SEQ ID No 3 or a sequence having at least 80%, 90%, 95%, or 99% identity thereof, as a β1,3 galactosyl transferase to catalyze the transfer of a galactose moiety from UDP-Gal to an acceptor bearing the terminal non reducing structure GalNAcβ-3-R to form the Galβ-3GalNAcβ-3-R structure, wherein R is selected from the group consisting of galactose, galactose-X, β galactosides such as allyl-β-galactoside or propargyl-β-galactoside, a galactosides, globotriose, β globotrioside such as allyl-β-globotrioside or propargyl-β-globotrioside, a globotrioside, X being defined as a reactive group allowing the covalent coupling with an other molecule, including amino, azide and nitrophenyl groups.

In another embodiment, the invention relates to the use of a microorganism comprising an heterologous lgtD gene from Haemophilus influenzae of SEQ ID No 3 or a sequence having at least 80% identity thereof to produce an oligosaccharide selected from Galβ-3GalNAcβ-3Gal, Galβ-3GalNAcβ-3Galα-X, Galβ-3GalNAcβ-3Galβ-X, Galβ-3GalNAcβ-3Galβ-allyl, Galβ-3GalNAcβ-3Galβ-propragyl, Galβ-3GalNAcβ-3Galα-4Galβ-4Gal (Globopentaose), Galβ-3GalNAcβ-3Galα-4Galβ-4Galα-X, Galβ-3GalNAcβ-3Galα-4Galβ-4Gal, —X, Galβ-3GalNAcβ-3Galα-4Galβ-4Galβ-allyl, and Galβ-3GalNAcβ-3Galα-4Galβ-4Galβ-propargyl.

| Example of produced structure | R = |
|---|---|
| Galβ-3GalNAcβ-3Gal | Gal |
| Galβ-3GalNAcβ-3Galα-X | α galactosides, |
| Galβ-3GalNAcβ-3Galβ-X | β galactosides, |
| Galβ-3GalNAcβ-3Galβ-allyl | allyl-β-galactoside |
| Galβ-3GalNAcβ-3Galβ-propragyl | propargyl-β- |
| | galactoside |
| Galβ-3GalNAcβ-3Galα-4Galβ-4Gal | globotriose |
| Galβ-3GalNAcβ-3Galα-4Galβ-4Galα-X | α globotrioside |
| Galβ-3GalNAcβ-3Galα-4Galβ-4Galβ-X | β globotrioside |
| Galβ-3GalNAcβ-3Galα-4Galβ-4Galβ-allyl | allyl-β-globotrioside |
| Galβ-3GalNAcβ-3Galα-4Galβ-4Galβ-propargyl | propargyl-β- |
| | globotrioside |

Thus, galactose can be supplied in the culture medium and is internalized by engineered micro-organisms and used as precursor.

In this regard, the invention contemplates a method comprising culturing a microorganism for producing:

Galβ-3GalNAcβ-3Gal (SSEA-3 antigen)

NeuAcα-3Galβ-3GalNAcβ-3Gal (SSEA-4 antigen)

Fucα-2Galβ-3GalNAcβ-3Gal (Globo-H antigen)

### DESCRIPTION OF THE DRAWINGS

FIG. 1 shows A) structures of globotriose and globotetraose and B) a strategy for metabolically engineered pathway for globotriose (1) and globotetraose (4) production from lactose in Escherichia coli K 12. Lactose is internalized by the β-galactoside permease LacY and accumulates in the cytoplasm because the strain is a lacZ mutant devoid of β-galactosidase activity. Using the endogenous UDP-Gal pool, the α-4 galactosyltransferase (LgtC) converts lactose into globotriose (1). The later cannot be degraded because the melA gene for α-galactosidase has been inactivated. Globotriose (1) can be further converted to globotetraose by the β-3GalNAc transferase encoded by lgtD. Since Escherichia coli K12 is not normally able to synthesize UDP-GalNAc, the strain was complemented with the Pseudomonas aeruginosa wbpP gene for UDP-GlcNAc-C4 epimerase.

FIG. 2 shows the production of globotriose (1) in a high-cell density culture of the TA19 strain. (▴) bacterial growth; (▪) extracellular lactose; (♦) intracellular lactose; (⋄) intracellular globotriose; (□) extracellular globotriose; (Δ) sum of intracellular and extracellular globotriose. The arrow indicates the start of induction and the addition of lactose (14.6 mM).

FIG. 3 shows the separation on Biogel P2 of the intracellular oligosaccharide fraction from (A) the culture of the globotriose producing TA19 strain (compounds 1, 2 and 3) and (B) the culture of the globotetraose-producing strain TA11 (compound 4).

FIG. 4 displays the metabolically engineered pathway for globotetraose production from exogenous globotriose in Escherichia coli K 12. Globotriose (1) is taken up by the β-galactoside permease LacY and converted to globotetraose (4) by β-3 GalNAc transferase encoded by lgtD. Since Escherichia coli K12 is not normally able to synthesize UDP-GalNAc, the strain was complemented with the Pseudomonas aeruginosa wbpP gene for UDP-GlcNAc-C4 epimerase.

FIG. 5 is TLC plate analysis of the oligosaccharide content in the intracellular fraction (lanes 3, 4, 5, 6) and in the extracellular fraction (lanes 7, 8, 9, 10) of samples withdrawn from culture of the globotetraose-producing TA21 strain immediately after the addition of globotriose and after 2, 4 and 6 hours of culture, respectively. Standards are in lane 1 (lactose) and in lanes 2 and 11 (globotriose).

FIG. 6 shows TLC analysis of oligosaccharides produced by high cell density culture of strain TA19. The initial lactose concentration was 7.5 g.l⁻¹. Lanes 1: standard solution (2 mg.ml⁻¹ each) of lactose lacto-N-neotetraose (LNnT), lacto-N-neohexaose (LNnH). Lanes 2, 3, 4, 5, 6, 7, 8: intracellular fractions withdrawn 0, 2, 3, 4, 5, 6 and 8 hours after lactose addition.

FIG. 7 is a chromatography on Biogel P2 of the Charcoal purified fraction from the 8 hours culture of stain TA 19.

FIG. 8 is a chromatography on Biogel P2 of intracellular fraction of culture of strain TA21 harvested 6 hours (A) or 20 hours (B) after the addition of globotriose. Peak 1 and 2 and 3 were identified to globotriose, globotetraose and globopentaose respectively.

FIG. 9 displays the metabolically engineered pathway for globopentaose production from exogenous globotriose in Escherichia coli K 12. Globotriose is taken up by the β-galactoside permease LacY and converted into globotetraose by the β-3 GalNAc transferase encoded by lgtD. Since Escherichia coli K12 is not normally able to synthesize UDP-GalNAc, the strain was complemented with the Pseudomonas aeruginosa wbpP gene for UDP-GlcNAc-C4 epimerase. The globotetraose is then converted into globopentaose by the β-3 Gal transferase activity encoded by lgtD.

FIG. 10 displays the metabolically engineered pathway for the production of the Globo-H hexasaccharide (Fucα-2Galβ-3GalNAcβ-3Galα-4Galβ-4Gal) from exogenous globotriose in Escherichia coli K 12. Globotriose is taken up by the β-galactoside permease LacY and converted into globotetraose by the β-3 GalNAc transferase encoded by lgtD. Since Escherichia coli K12 is not normally able to synthesize UDP-GalNAc, the strain was complemented with the Pseudomonas aeruginosa wbpP gene for UDP-GlcNAc-C4 epimerase. The globotetraose is then converted into globopentaose by the β-3 Gal transferase activity encoded by lgtD. The globopentaose is finally converted into the Globo-H hexasaccharide by the α-2 fucosyltranferase encoded by futC.

FIG. 11. TLC analysis of oligosaccharides produced by high cell density culture of strain MR20. The initial globotriose concentration was 3 g.l⁻¹. Lane 4: standard solution of Globotriose, Globotetraose and Globopentaose. Lanes 8: standard solution (2 mg.ml⁻¹ each) of lactose, lacto-N-neotetraose (LNnT) lacto-N-neohexaose (LNnH). Lanes 1, 2, 3, 5, 6, and 7: intracellular fractions withdrawn 3, 7, 23, 28, 31 and 47 hours after globotriose addition.

FIG. 12 shows the metabolically engineered pathway for the production of the SGG hexasaccharide (NeuAcα-3Galβ-3GalNAcβ-3Galα-4Galβ-4Gal) from exogenous globotriose and NeuAc in Escherichia coli K 12. Globotriose is taken up by the β-galactoside permease LacY and converted into globotetraose by the β-3 GalNAc transferase encoded by lgtD. Since Escherichia coli K12 is not normally able to synthesize UDP-GalNAc, the strain was complemented with the Pseudomonas aeruginosa wbpP gene for UDP-GlcNAc-C4 epimerase. The globotetraose is then converted into globopentaose by the β-3 Gal transferase activity encoded by lgtD. The globopentaose is finally converted into the SGG hexasaccharide by the Neisseria α-3 sialyltansferase. CMP-NeuAc is produced from exogenous NeuAc which is taken up by the NanT permease. To prevent the catabolism of NeuAc, a mutant nanA⁻ strain devoid of NeuAc aldolase activity is used.

FIG. 13 Use of galactose as acceptor for the production of the terminal structure of globo-H. The strain MR17 was constructed by transforming the GLK host strain with the three plasmids pBS-lgtD-gne, pBBRGAB and pWKS-lgtC to produce the terminal tetrasaccharide epitope of the Globo-H antigen as illustrated in FIG. 13. Culture of strain MR17 in presence of 3 g.l⁻¹ of galactose led to the formation of major compound (5) that migrated in TLC as a tetrasaccharide (FIG. 14). Compound (5) was purified by adsorption on activated charcoal and size exclusion chromatography with a yield of 1.66 gram starting from a one liter culture. Identification of (5) as Fucα-2Galβ-3GalNAcβ-3Gal was confirmed by mass spectrometry and NMR analysis. Significant amount of a compound (2) that migrated slower than galactose (1) but faster than lactose was recovered in both the intra and extra cellular fraction and was identified as the disaccharide Fucα-2Gal. Two other compounds (3) and (4) transiently accumulated and were identified to the disaccharide GalNAcβ-3Gal and the trisaccharide Galβ-3GalNAβ-3Gal by their migration rate in TLC and their mass spectrometry data.

FIG. 14. TLC analysis of oligosaccharides produced by high cell density culture of strain MR17 (GLK, pBS-lgtD-gne, pBBR-GAB, pWKS-futC). The initial galactose concentration was 3 g.l⁻¹. Lanes 1 and 6: standard solution (2 mg.ml⁻¹ each) of lactose, lacto-N-neotetraose (LNnT) lacto-N-neohexaose (LNnH) and globotriose Lane 11 standard solution of the trisaccharide Galβ-3GalNAcβ-3Gal. Lanes 2, 3, 4, and 5: extracellular fractions withdrawn 0, 3, 7, and 22 hours after galactose addition. Lanes 7, 8, 9 and 10: intracellular fractions withdrawn 0, 3, 7, and 22 hours after galactose addition.

### DETAILED DESCRIPTION OF THE INVENTION

In a first embodiment, the invention relates to a method for producing an oligosaccharide comprising the galabiose motif (Galα-4Gal), herein referred to as globosides oligosaccharides or globosides sugars or “globosides”, the method comprising culturing a (first) microorganism in a culture medium comprising lactose, wherein said (first) microorganism comprises a heterologous gene encoding α-1,4-Gal transferase (e.g., a lgtC gene) which transfers a galactose moiety from UDP-Gal to the lactose to form globotriose (Galα-4Galβ-Glc) and wherein lactose is in excess in the culture medium.

Indeed, we show hereafter that globotriose (1) can be produced in high yields (7 g.l⁻¹) by bacterial fermentation. However, characterization of the oligosaccharide fraction revealed that a series of galactosylated derivatives of globotriose may also be synthesized at the end of the fermentation time course. With excess lactose in the culture, however, we didn't observe any polygalactosylation until the complete consumption of lactose had been reached. Thus, polygalactosylation can be avoided by terminating the culture before the exhaustion of lactose. Furthermore, our conditions allow the recovery of most of the globotriose in the extracellular medium, because the presence of lactose prevents the re-entry of globotriose which has rapidly diffused outside the cells. Thus, in the method described above the α-1,4-Gal transferase enzyme can be encoded for example by LgtC genes of N. meningitidis, N. gonorrhoeae or Haemophilus influenzae, more particularly by the LgtC gene of Neisseria meningititis L1 (126E) GenBank accession number U65788—SEQ ID No 1, protein_id AAB48385—SEQ ID No 2). One of skill will recognize that enzymes that are encoded by nucleic acids that are at least substantially identical (as defined below) to the exemplified sequences can also be used.

The invention also relates to a microorganism comprising a heterologous gene encoding α-1,4-Gal transferase and which is engineered to enhance the efficiency of the claimed methods. Typically, the microorganism preferrably encodes a protein that facilitates uptake of lactose and lacks enzymes that metabolize lactose. For example, in E. coli, the cell is preferrably LacY+ (β-galactoside permease), LacZ− (β galactosidase), and MelA− (α-galactosidase). The invention also relates to a cell culture medium comprising lactose in excess and the above microorganism.

Globotriose High Yield and Specific Production

Depending on the endpoints and in one specific aspect, the method as depicted above may further include terminating the culture before the exhaustion of lactose and extracting globotriose molecules from the extracellular medium. Besides, increasing the concentration to reach from 5 g.L−1 to 10 g.L−1 lactose, preferably about 7.5 g.L−1 prevent the formation of polygalactosylated side-products such as polygalactosylated tetrasaccharide (Galα-4Galα-4Galβ-4Gal) and pentasaccharide (Galα-4Galα-4Galα-4Galβ-4Gal). This results in significant improvement of globotriose yield. Advantageously, the culture can be terminated at about 8 hours after lactose addition to prevent a further galactosylation of globotriose.

Optionally, this method may further include a separation and/or purifying step to recover globotriose molecules from the medium. A concentration or lyophilization step may also be included to prepare a globotriose composition such as a solution or a dry product suitable for different utilities or as a commodity. As explained before, purification steps allow one to prepare such solution or dry product with high globotriose purity, such as more than 80%, 85%, 90%, 95% or even 99% globotriose by weight of the total composition. The invention is thus aimed at a composition of pure globotriose obtained by the above method.

Globotriose and Globotetraose Production in a One-Step Fermentation Process

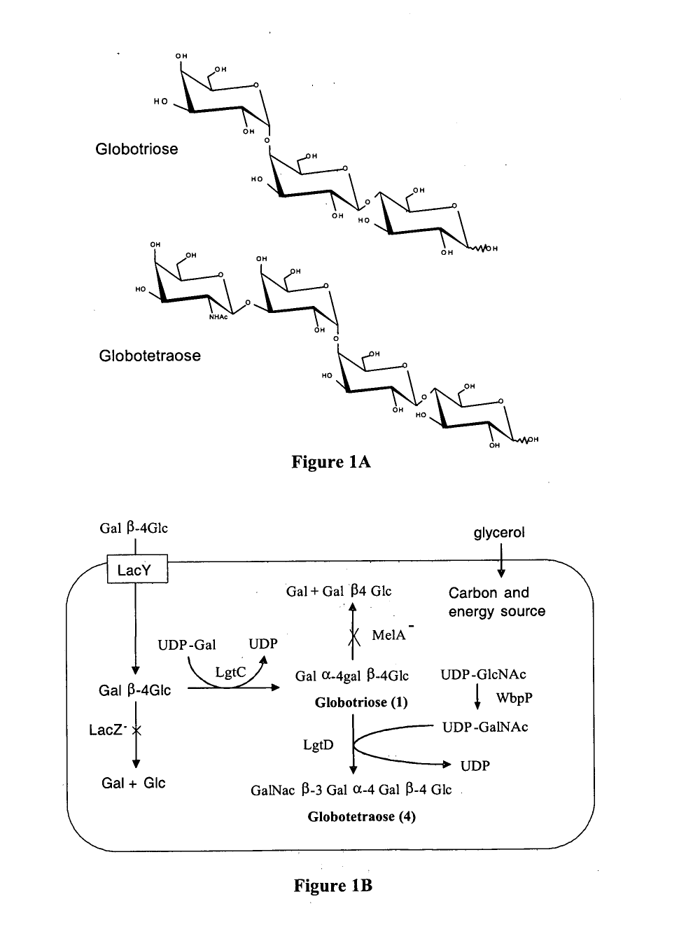

Also encompassed herein is a method as defined above for producing oligosaccharides comprising the galabiose motif (Galα-4Gal), herein referred as globosides, the method comprising culturing a microorganism in a culture medium comprising excess lactose, wherein said microorganism comprises a heterologous gene encoding α-1,4-Gal transferase (e.g., a lgtC gene) which transfers a galactose moiety from UDP-Gal to the lactose to form globotriose (Galα-4Galβ-4Glc) and a heterologous gene encoding β-3 GalNAc transferase (e.g., a lgtD gene) which transfers a GalNAc moiety from UDP-GalNAc to the globotriose to form globotetraose (GalNAcβ-3Galα-4Galβ-4Glc). Advantageously, this microorganism also comprises a gene encoding for UDP-GlcNAc-C4 epimerase, such as the Pseudomonas aeruginosa wbpP gene, or a gne gene encoding for a UDP-glucose 4-epimerase, such as the gne gene of C. jejuni strain NCTC 11168 of SEQ ID No 9. In addition, this microorganism is advantageously LacY+ (β-galactoside permease) and LacZ− as well as melA−, meaning that the melA gene for α-galactosidase activity (melibiase) had been inactivated [21]. In a particular embodiment, the micoorganism is an E. coli K12 strain JM107 derivative (ATCC 47014). This particular method is basically as shown in FIG. 1B. In some embodiments, the invention also relates to a microorganism, for example, an E. coli comprising a heterologous lgtC gene encoding α-1,4-Gal transferase and a heterologous lgtD gene encoding β-3 GalNAc transferase and which is LacY+ (β-galactoside permease), LacZ− (β galactosidase), and MelA− (α-galactosidase). This microorganism comprises advantageously a wbpP gene encoding for UDP-GlcNAc-C4 epimerase or a gne gene encoding for a UDP-glucose 4-epimerase, such as the gne gene of C. jejuni strain NCTC 11168 of SEQ ID No 9. It also relates to a cell culture medium comprising lactose in excess and the above microorganism.

Globotretraose Production Coupled with Globotriose Production—a Two-Step Fermentation Process

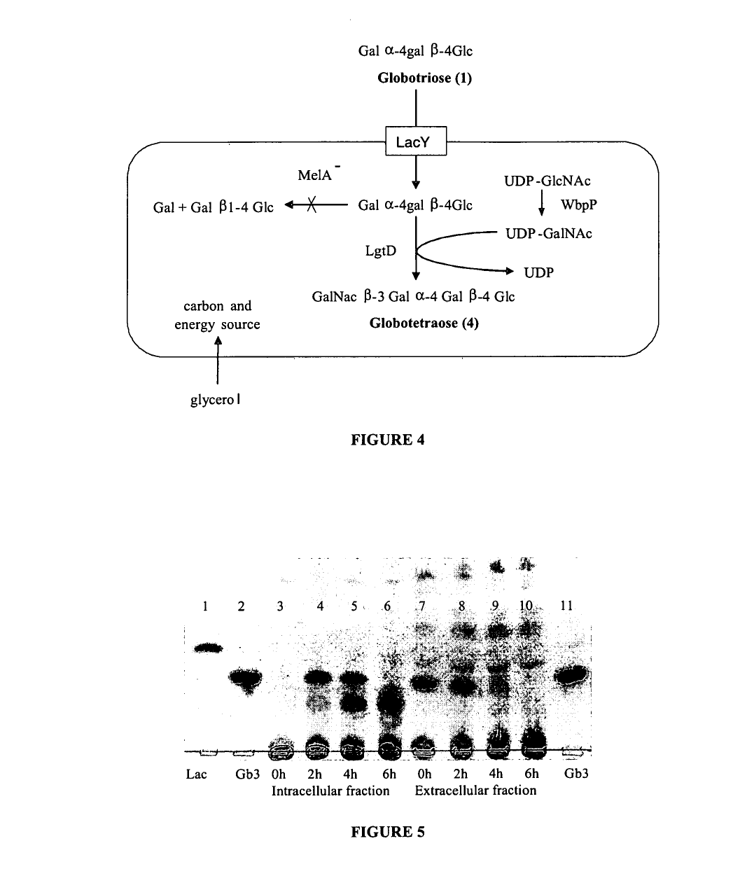

Depending on the endpoints and in another specific aspect, the method as depicted above may further include terminating the culture before the exhaustion of lactose, using or extracting globotriose molecules in the extracellular medium and providing said globotriose molecules as a precursor for producing globotretraose; the method further comprising culturing a second microorganism in a culture medium comprising globotriose, wherein said second microorganism comprises a heterologous a gene encoding β-3 GalNAc transferase (e.g., a lgtD gene) which transfers a GalNAc moiety from UDP-GalNAc to the globotriose to form globotetraose (GalNAcβ-3Galα-4Galβ-4Glc). Advantageously, said second microorganism also comprises a gene encoding for UDP-GlcNAc-C4 epimerase, such as the Pseudomonas aeruginosa wbpP gene or a gne gene encoding for a UDP-glucose 4-epimerase, such as the gne gene of C. jejuni strain NCTC 11168 of SEQ ID No 9. This particular embodiment is illustrated in FIG. 4. In addition, both first and second microorganisms are typically engineered to enhance the efficiency of the claimed methods. Thus, the microorganisms preferrably encode a protein that facilitates uptake of lactose and lack enzymes that metabolize lactose. For example in E. coli, the cell is advantageously LacY+ (β-galactoside permease) and LacZ−. Besides, these microorganisms are preferably engineered to lack α-galactosidase activity. In E. coli, the cell is typically melA−, meaning that the melA gene for α-galactosidase activity (melibiase) had been inactivated [21]. In a particular embodiment, the micoorganism is an E. coli K12 strain JM107 derivative (ATCC 47014). It is also preferred to add glycerol in the medium as carbon and energy source.

The invention also relates in some embodiments to said second microorganism (e.g., an E. coli which is LacY+, LacZ−, MelA−) comprising a heterologous a lgtD gene (β-3 GalNAc transferase) and a heterologous wbpP gene (UDP-GlcNAc-C4 epimerase), such as the Pseudomonas aeruginosa wbpP gene or a gne gene encoding for a UDP-glucose 4-epimerase, such as the gne gene of C. jejuni strain NCTC 11168 of SEQ ID No 9 and to a cell culture medium comprising the above microorganism and globotriose for example at a concentration of 1 to 10 g.L⁻¹.

Here, the invention also contemplates in some embodiments a set of two separate microorganisms, the first microorganism being LacY+, LacZ−, and MelA− and comprising a heterologous lgtC gene; the second being LacY+, MelA− and comprising a heterologous lgtD gene, and a heterologous wbpP gene or a gne gene encoding for a UDP-glucose 4-epimerase, such as the gne gene of C. jejuni strain NCTC 11168 of SEQ ID No 9.

This method coupling first the production of globotriose with said first microorganism as defined above, then producing giobotetraose and other globosides using said second microorganism as mentioned above is possible because globotriose is in fact internalized by an active process which is probably be mediated by, for example, a β-Galactoside permease expressed in our system. β-galactoside permease has a broad substrate specificity and has been shown to internalize α-galactosides such as the trisaccharide raffinose [24]. However it is also possible that Globotriose is internalized by other E; coli sugar permease such as the melibiose transporter encoded by the melB gene. Nevertheless, globotriose internalization came as a surprise since we had already observed that short oligosaccharides such as the trisaccharide LNT2 diffused rapidly in the extracellular medium [17] and that no re-entry into the cells was observed. Thus, depending on the endpoint, a convenient way to avoid the production of multiple side-products during globotetraose synthesis is to use globotriose as the acceptor instead of lactose, as described in the culture of the TA21 strain (see examples below). Besides, increasing the concentration to reach from 5 g.L−1 to 10 g.L−1 lactose, preferably about 7.5 g.L−1 prevent the formation of polygalactosylated side-products such as polygalactosylated tetrasaccharide (Galα-4Galα-4Galβ-4Gal) and pentasaccharide (Galα-4Galα-4Galα-4Galβ-4Gal). This results in significant improvement of globotriose yield. Advantageously, the culture can be terminated at about 8 hours after lactose addition to prevent a further galactosylation of globotriose.

Globotetraose production can thus be envisioned as a two-step fermentation process. In the first step, globotriose is produced from lactose under conditions that limit the polygalactosylation reaction and favor extracellular globotriose accumulation as explained above. The extracellular globotriose is then separated from the globotriose producing cells and added to the globotetraose-producing cells.

In addition, we have extended this process to the synthesis of more complex carbohydrate structures of the globo series of glycosphingolipids as shown below, such as the Forsmann antigen, the stage-specific embryonic antigen 4 (SSEA-4) and Globo-H, that are involved in important developmental and pathological processes.

Globopentaose Production

In this regard, the invention is directed to a method for producing globopentaose (11) comprising culturing a microorganism in a culture medium comprising globotriose, wherein said microorganism comprises a heterologous a gene encoding β-3 GalNAc transferase (e.g., a lgtD gene) which transfers a GalNAc moiety from UDP-GalNAc to the globotriose to form globotetraose (GalNAcβ-3Galα-4Galβ-4Glc) and extending the culture to allow said β-3 GalNAc transferase to transfer a galactose moiety from UDP-Gal to globotetraose to form globopentaose (Galβ-3GalNAcβ-3Galα-4Galβ-4Gal). Advantageously, this microorganism also comprises a gene encoding for UDP-GlcNAc-C4 epimerase, such as the Pseudomonas aeruginosa wbpP gene or a gne gene encoding for a UDP-glucose 4-epimerase, such as the gne gene of C. jejuni strain NCTC 11168 of SEQ ID No 9. This particular embodiment is illustrated in FIG. 9. The invention is also directed to a set of two separate microorganisms, comprising said first microorganism as defined above and said second microorganism, wherein said second microorgansism is engineered to enhance the efficiency of the claimed methods. Thus, the microorganisms preferrably encode a protein that facilitates uptake of lactose and lack enzymes that metabolize lactose. For example in E. coli, the cell is LacY+, LacZ−, melA− and comprises, for example, a heterologous a lgtD gene and a wbpP gene, such as the Pseudomonas aeruginosa wbpP gene or a gne gene encoding for a UDP-glucose 4-epimerase, such as the gne gene of C. jejuni strain NCTC 11168 of SEQ ID No 9. The invention also concerns the use of a gene encoding β-3 GalNAc transferase, for example, the lgtD gene from Haemophilus influenzae H11578, GenBanK accession number U32832-SEQ ID No 3, protein_id=AAC23227—SEQ ID No 4, to catalyze the transfer of a galactose moiety from UDP-Gal to globotetraose to form globopentaose (β-3 Gal transferase activity). One of skill will recognize that enzymes that are encoded by nucleic acids that are at least substantially identical (as defined below) to the exemplified sequences can also be used.

Globo-H Production

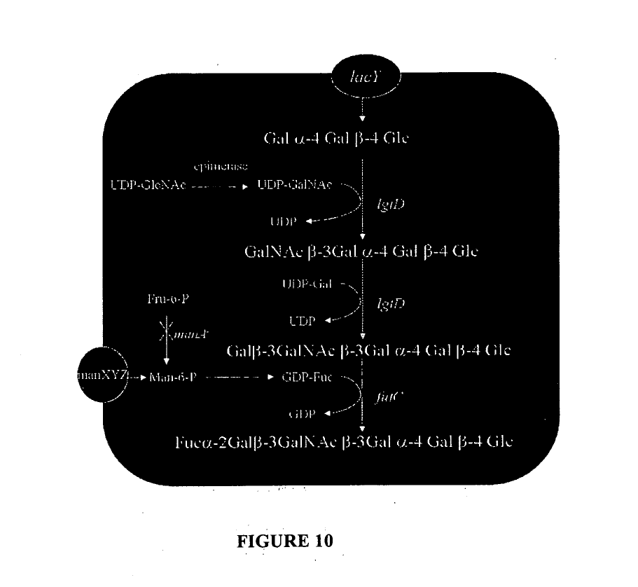

In still another embodiment, the invention is aimed at a method for producing Globo-H hexasaccharide (12) comprising culturing a microorganism in a culture medium comprising globotriose, wherein said microorganism comprises a heterologous gene encoding β-3 GalNAc transferase which transfers a GalNAc moiety from UDP-GalNAc to globotriose to form globotetraose (GalNAcβ-3Galα-4Galβ-4Glc), extending the culture to allow said 1-3 GalNAc transferase to transfer a galactose moiety from UDP-Gal to globotetraose to form globopentaose (Galβ-3GalNAcβ-3Galα-4Galβ-4Gal) and wherein said microorganism further comprises a heterologous gene encoding an α-2 fucosyltranferase (e.g., a futC gene) to transfer a fucose moiety from GDP-Fuc to globopentaose to form Globo-H hexasaccharide (Fucα-2Galβ-3GalNAcβ-3Galα-4Galβ-4Gal). Advantageously, this microorganism also comprises a gene encoding for UDP-GlcNAc-C4 epimerase, such as the Pseudomonas aeruginosa wbpP gene or a gne gene encoding for a UDP-glucose 4-epimerase, such as the gne gene of C. jejuni strain NCTC 11168 of SEQ ID No 9. In one particular embodiment, the strain may be a mutant engineered so that it is unable to synthesize GDP-Fuc, unless mannose is exogenously added. For example, an E. coli which is manA⁻ devoid of phophomannose isomerase activity and manXYZ+. Indeed, this mutant will be unable to synthesize GDP-Fuc, unless mannose is exogenously added in the medium and taken up and phosphorylated in Man-6-P by the mannose permease encoded by the manXYZ genes. By adding the mannose after the entire conversion of globotriose into globopentaose, the fucosylation of globotriose will be impossible and all the globotriose will be converted into Globo-H oligosaccharide. This particular embodiment is illustrated in FIG. 10. In another embodiment, since we observed that fucosyltransferase is not very active on globotriose, this strain may simply be LacY+, MelA−. In this regard, the invention contemplates said second microorganism which is LacY+, manA⁻ and which comprises a heterologous a lgtD gene (β-3 GalNAc transferase), a heterologous wbpP gene (UDP-GlcNAc-C4 epimerase), such as the Pseudomonas aeruginosa wbpP gene or a gne gene encoding for a UDP-glucose 4-epimerase, such as the gne gene of C. jejuni strain NCTC 11168 of SEQ ID No 9 and a heterologous futC gene (α-2 fucosyltranferase), such as the Helicobacter pylori gene futC of SEQ ID No 5. Thus, the MelA− and manXXZ+ features are optional as well as the addition of mannose.

The invention also relates to a microorganism, which in some embodiments is LacY+, MelA−, manXYZ+, and optionally manA⁻; comprising, for example, a heterologous lgtD gene (β-3 GalNAc transferase), a heterologous wbpP gene (UDP-GlcNAc-C4 epimerase), such as the Pseudomonas aeruginosa wbpP gene or a gne gene encoding for a UDP-glucose 4-epimerase, such as the gne gene of C. jejuni strain NCTC 11168 of SEQ ID No 9 and a heterologous futC gene (α-2 fucosyltranferase), such as the Helicobacter pylori gene futC from strain UA802 (GenBank accession number AF076779—SEQ ID No 5, protein_id=AAC99764—SEQ ID No 6) or from strain 26695 (HP0094 and HP0093, GenBank AE000531) to a cell culture medium comprising the above microorganism, globotriose (for example 1 to 10 g.L⁻¹) and mannose (for example 0.35 to 3.5 g.L⁻¹). One of skill will recognize that enzymes that are encoded by nucleic acids that are at least substantially identical (as defined below) to the exemplified sequences can also be used.

Here, the invention also contemplates a set of two separate micoorganisms, the first microorganism being LacY+, LacZ−, and MelA− and comprising a heterologous lgtC gene; the second being LacY+, MelA−, manXXZ+, manA⁻ and comprising heterologous lgtD, wbpP (or gne), and futC genes.

Sialosyl Galactosyl Globoside Production

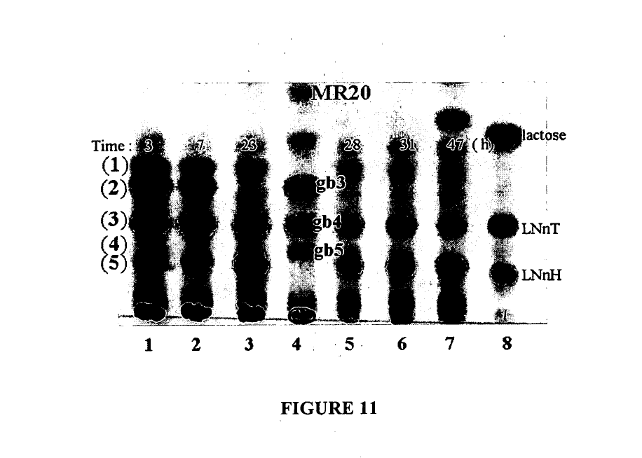

In still another embodiment, the invention is aimed at a method for producing sialosyl galactosyl globoside (SGG) hexasaccharide (13) comprising culturing a microorganism in a culture medium comprising globotriose, wherein said microorganism comprises a heterologous a gene encoding β-3 GalNAc transferase (a lgtD gene) which transfers a GalNAc moiety from UDP-GalNAc to the globotriose to form globotetraose (GalNAcβ-3Galα-4Galβ-4Glc), extending the culture to allow said β-3 GalNAc transferase to transfer a galactose moiety from UDP-Gal to the globotetraose to form globopentaose (Galβ-3GalNAcβ-3Galα-4Galβ-4Gal) and wherein said microorganism comprises a gene for the CMP-NeuAc synthase (for example from N. meningitidis) and a heterologous gene encoding α-3 sialyltransferase (for example from N. meningitidis, such the MC58 strain: GenBanK accession number U60660—SEQ ID No 7, protein_id=AAC44541.1—SEQ ID No 8) catalyzes the transfer of a sialyl moiety from an activated sialic acid molecule to globopentaose to form sialosyl galactosyl globoside (SGG) hexasaccharide (NeuAcα-3Galβ-3GalNAcβ-3Galα-4Galβ-4Gal). One of skill will recognize that enzymes that are encoded by nucleic acids that are at least substantially identical (as defined below) to the exemplified sequences can also be used. Advantageously, this microorganism also comprises a wbpP gene encoding for UDP-GlcNAc-C4 epimerase, such as the Pseudomonas aeruginosa wbpP gene or a gne gene encoding for a UDP-glucose 4-epimerase, such as the gne gene of C. jejuni strain NCTC 11168 of SEQ ID No 9. It is also advantageous that the strain is a mutant nanA− so as to be devoid of Neu5Ac aldolase activity and NanT+ allowing active transport of sialic acid. This particular embodiment is illustrated in FIG. 11.

The invention also relates to a microorganism, which in preferred embodiments is LacY+, MelA−, nanT+, nanA⁻ comprising, for example, a heterologous a lgtD gene (β-3 GalNAc transferase), a heterologous wbpP gene (UDP-GlcNAc-C4 epimerase), such as the Pseudomonas aeruginosa wbpP gene or a gne gene encoding for a UDP-glucose 4-epimerase, such as the gne gene of C. jejuni strain NCTC 11168 of SEQ ID No 9 and a heterologous gene for α-3 sialyltransferase, such as the gene from N. meningitis strain MC58 It also relates to a cell culture medium comprising the above microorganism, globotriose (for example 1 to 10 g.L⁻¹) and sialic acid (for example from 0.6 to 6 g.L⁻¹).

Here, the invention also contemplates a set of two separate micoorganisms, the said first microorganism being as mentioned above LacY+, LacZ−, and MelA− and comprising a heterologous lgtC gene; the second being LacY+, MelA−, nanT+, nanA⁻ and comprising heterologous lgtD, wbpP (or gne) and nst genes.

It will be understood herein that the extracellular globotriose source used in the above methods for producing globosides such as globotetraose, globopentaose, globo-H, and sialosyl galactosyl globoside (SGG) hexasaccharide may originate from culturing said first microorganism as described above (coupling system) which produces globotriose or in still another embodiment from any other source (non-coupling system). Thus, is embraced by the invention the method described above for producing globotetraose, globopentaose, globo-H, sialosyl galactosyl globoside (SGG) hexasaccharide wherein it comprises a first step using the said first microorganism which is LacY+, LacZ−, and MelA− and which comprises a heterologous lgtC gene. In other words, a set of microorganisms can be used. Alternatively, it is also within the invention to directly provide globotriose from any other source to the culture medium of said second micoorganism. In this regard, the invention encompasses a method for producing an oligosaccharide comprising the galabiose motif (Galα-4Gal), referred as globosides, selected the group consisting of globotetraose, globopentaose, and galactosyl-globosides including globo-H hexasaccharide, sialosyl galactosyl globoside (SGG) hexasaccharide, disialosyl galactosyl globoside comprising the step consisting of culturing a said second microorganism as defined above in a medium comprising globotriose. The invention also concerns a culture medium comprising globotriose preferably at a concentration ranging from 1 to 10 g.L⁻¹ and the use of the said first micoorganism and methods thereof to prepare a culture medium comprising globotriose. It is also within the scope to produce commercial scale compositions of the above globosides, such as a composition comprising one or several globoside(s) selected from the group consisting of globotriose, globotetraose, globopentaose, and galactosyl-globosides including globo-H hexasaccharide, and sialosyl galactosyl globoside (SGG) hexasaccharide.

In still another embodiment, the invention relates to the use of a LgtD gene encoding a GalNAc transferase, in particular the lgtD from H. influenzae (SEQ ID No 3) to transfer a GalNAc residue to galactose to form the intermediate GalNAcβ-3Gal and to produce oligosacharrides comprising GalNAcβ-3Gal. It also relates to the use of a LgtD gene encoding a GalNAc transferase, in particular the lgtD from H. influenzae (SEQ ID No 3) as a Gal transferase in presence of GalNAcβ-3Gal to form the terminal trisaccharide structure of the SSEA-3 antigen (Galβ-3GalNAcβ-3Gal). It also relates to a method for producing an oligosaccharide comprising the motif GalNAcβ-3Gal, the method comprising culturing a microorganism which is galP (galactose permease), LacZ− (D galactosidase), MelA− (α-galactosidase) and wbpP encoding for UDP-GlcNAc-C4 epimerase, such as the Pseudomonas aeruginosa wbpP gene; or a gne gene encoding for a UDP-glucose 4-epimerase, such as the gne gene of C. jejuni strain NCTC 11168 of SEQ ID No 9, in a culture medium comprising galactose, wherein said microorganism comprises a heterologous lgtD gene encoding α-1,4-Gal transferase which transfers a GalNAc residue to galactose to form the an oligosaccharide comprising GalNAcβ-3Gal. In this method, the lgtD gene can be allowed to further transfer a galactose moiety from UDP-Gal to GalNAcβ-3Gal to form the terminal trisaccharide structure of the SSEA-3 antigen (Galβ-3GalNAcβ-3Gal). This method can be extended to the production of the terminal tetrasaccharide structure of the SSEA-4 antigen (NeuAcα-3Galβ-3GalNAcβ-3Gal). In this regard, the microorganism further comprises a heterologous gene encoding an α-3-sialyltransferase to transfer a sialic acid moiety from CMP-NeuAc to Galβ-3GalNAcβ-3Gal to form NeuAcα-3Galβ-3GalNAcβ-3Gal. The invention contemplates the above microorganism to produce GalNAcβ-3Gal, Galβ-3GalNAcβ-3Gal and NeuAcα-3Galβ-3GalNAcβ-3Gal as well as a culture medium comprising galactose and said microorganism.

This method can be extended to the production of the terminal tetrasaccharide structure of the Globo-H antigen (Fucα-2Galβ-3GalNAcβ-3Gal). In this regard, the microorganism further comprises a heterologous futC gene encoding an α-2 fucosyltranferase to transfer a sialic acid moiety from GDP-Fuc to Galβ-3GalNAcβ-3Gal to form Fucα-2Galβ-3GalNAcβ-3Gal.

The nomenclature and general laboratory procedures required to practice the present invention are well known to those of skill in the art. These procedures can be found, for example, in Sambrook, et al., Molecular Cloning: A Laboratory Manual (2nd Ed.), Vol. 1-3, Cold Spring Harbor Laboratory, Cold Spring Harbor, N.Y., 1989.

Definitions

An “acceptor substrate” or an “acceptor saccharide” for a glycosyltransferase is an oligosaccharide moiety that can act as an acceptor for a particular glycosyltransferase. When the acceptor substrate is contacted with the corresponding glycosyltransferase and sugar donor substrate, and other necessary reaction mixture components, and the reaction mixture is incubated for a sufficient period of time, the glycosyltransferase transfers sugar residues from the sugar donor substrate to the acceptor substrate. For example, an acceptor substrate for the production of globotetraose, globopentaose, globo-H, sialosyl galactosyl globoside (SGG) hexasaccharide and disialosyl galactosyl globoside using heterologous lgtD, futC and/or nst in the methods of the invention is globotriose.

A “donor substrate” for glycosyltransferases is an activated nucleotide sugar. Such activated sugars generally consist of uridine, guanosine, and cytidine monophosphate derivatives of the sugars (UMP, GMP and CMP, respectively) or diphosphate derivatives of the sugars (UDP, GDP and CDP, respectively) in which the nucleoside monophosphate or diphosphate serves as a leaving group. For example, a donor substrate for α-2 fucosyltranferase used in the methods of the invention is GDP-Fuc.

A “culture medium” refers to any liquid, semi-solid or solid media that can be used to support the growth of a microorganism used in the methods of the invention. In some embodiments, the microorganism is a bacteria, e.g., E. coli. Media for growing microorganisms are well known, see, e.g., Sambrook et al. and Current Protocols in Molecular Biology, F. M. Ausubel et al., eds., Current Protocols, a joint venture between Greene Publishing Associates, Inc. and John Wiley & Sons, Inc., (1998 Supplement) (Ausubel). Media can be rich media, e.g., Luria broth or terrific broth, or synthetic or semi-synthetic medium, e.g., M9 medium. In some preferred embodiments the growth medium comprises either lactose or globotriose as well as mannose or sialic acid.

“Commercial scale” refers to gram scale production of a sialylated product saccharide in a single reaction. In preferred embodiments, commercial scale refers to production of greater than about 50, 75, 80, 90 or 100, 125, 150, 175, or 200 grams.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

A “heterologous polynucleotide” or a “heterologous gene”, as used herein, is one that originates from a source foreign to the particular host cell, or, if from the same source, is modified from its original form. Thus, a heterologous sialyltransferase gene in a cell includes a gene that is endogenous to the particular host cell but has been modified. Modification of the heterologous sequence may occur, e.g., by treating the DNA with a restriction enzyme to generate a DNA fragment that is capable of being operably linked to a promoter. Techniques such as site-directed mutagenesis are also useful for modifying a heterologous sequence.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used. Transcription termination signals, enhancers, and other nucleic acid sequences that influence gene expression, can also be included in an expression cassette. When more than one heterologous protein is expressed in a microorganism, the genes encoding the proteins can be expressed on a single expression cassette or on multiple expression cassettes that are compatible and can be maintained in the same cell. As used herein, expression cassette also encompasses nucleic acid constructs that are inserted into the chromosome of the host microorganism. Those of skill are aware that insertion of a nucleic acid into a chromosome can occur, e.g., by homologous recombination. An expression cassette can be constructed for production of more than one protein. The proteins can be regulated by a single promoter sequence, as for example, an operon. Or multiple proteins can be encoded by nucleic acids with individual promoters and ribosome binding sites.

Non limitative examples of genes and plasmids used herein are depicted in Table 1 below:

Table 1: Substrate specificity of lgtD protein. Kinetic parameter for UDP-GalNAc and UDP-Gal in presence of globotriose or globotetraose as acceptor.

TABLE 1
Genes, plasmids and Escherichia coli
strains used in present invention

Plasmids and
strains

| | | |
|---|---|---|
| genes | | |
| lgtD | GalNAc transferase from | SEQ ID No 3 |
| | H. influenzae | |
| gne Cj1131c | UDP-glucose epimerase from | CAB73386.1 |
| | C. jejuni NTCC1168 | S̅E̅Q̅ I̅D̅ N̅o̅ 9̅ |
| futC | Helicobacter pylori fucosyl- | SEQ ID No 5 |
| | transferase | |
| gmd, wcaG, | E. coli genes coding GDP-Man | GenBanK |
| manC manB | dehydratase, fucose synthase, | U38473 |
| | GDP-Man pyrophosphorylase and | |
| | Phosphomannomutase | |
| | respectively | |
| plasmids | | |
| pBS-lgtD-gne | pBluescript II SK derivative | present |
| | carrying lgtD and gne | invention |
| pWKS130 | Cloning vector, Km^{r}, Plac | (Wang & |
| | promoter, low copy number, | Kushner, 1991) |
| | pSC101 replicon | |
| pWKS-lgtC | pWKS130 derivative carrying | present |
| | futC | invention |
| pBBRGAB | pBBR1MCS-3 derivative carrying | (Dumon et al., |
| | gmd, wcaG, manC and manB | 2006) |
| strains | | |
| DC | DH1 lacZ lacA | (Dumon et al., |
| | | 2006) |
| DM | DC melA | present |
| | | invention |
| MR15 | DM (pBS-lgtD-gne) | present |
| | | invention |
| MR20 | DM (pBS-lgtD-gne, pBBRGAB, | present |
| | pWKS-lgtC) | invention |
| GLK | DC galK | (Dumon et al., |
| | | 2006) |
| MR16 | GLK (pBS-lgtD-gne) | present |
| | | invention |
| MR17 | GLK (pBS-lgtD-gne, pBBRGAB, | present |
| | pWKS-lgtC) | invention |

The term “isolated” refers to material that is substantially or essentially free from components which interfere with the activity biological molecule. For cells, saccharides, nucleic acids, and polypeptides of the invention, the term “isolated” refers to material that is substantially or essentially free from components which normally accompany the material as found in its native state. Typically, isolated saccharides, oligosaccharides, proteins or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, e.g., sialylated products, purity can be determined using, e.g., thin layer chromatography, HPLC, NMR or mass spectroscopy.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

As noted above, those of skill will recognize that polynucleotides or polypeptides that are at least substantially identical to those exemplified here can be used in the present invention. The phrase “substantially identical,” in the context of two nucleic acids or polypeptides, refers to two or more sequences or subsequences that have at least 60%, preferably 80% or 85%, most preferably at least 90%, 91%, 92%, 93%, 94%, 95%, 96%, 97%, 98%, or 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids

Res. 25: 3389-3402, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode the enzymes are preferably optimized for expression in a particular host cell (e.g., yeast, mammalian, plant, fungal, and the like) used to produce the enzymes.

Similarly, “conservative amino acid substitutions,” in one or a few amino acids in an amino acid sequence are substituted with different amino acids with highly similar properties are also readily identified as being highly similar to a particular amino acid sequence, or to a particular nucleic acid sequence which encodes an amino acid. Such conservatively substituted variations of any particular sequence are a feature of the present invention. Individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. See, e.g., Creighton (1984) Proteins, W.H. Freeman and Company.

Host Cells

The microorganisms referred herein to practice the invention are recombinant cells. Recombinant cells are generally made by creating or otherwise obtaining a polynucleotide that encodes the particular enzyme(s) of interest, placing the polynucleotide in an expression cassette under the control of a promoter and other appropriate control signals, and introducing the expression cassette into a cell. More than one of the enzymes can be expressed in the same host cells using a variety of methods. For example, a single extrachromosomal vector can include multiple expression cassettes or more that one compatible extrachromosomal vector can be used maintain an expression cassette in a host cell. Expression cassettes can also be inserted into a host cell chromosome, using methods known to those of skill in the art. Those of skill will recognize that combinations of expression cassettes in extrachromosomal vectors and expression cassettes inserted into a host cell chromosome can also be used. Other modification of the host cell, described in detail below, can be performed to enhance production of the desired oligosaccharide. For example, the microorganism may be LacY+ allowing active transport of lactose.

The recombinant cells of the invention are generally microorganisms, such as, for example, yeast cells, bacterial cells, or fungal cells. Examples of suitable cells include, for example, Azotobacter sp. (e.g., A. vinelandii), Pseudomonas sp., Rhizobium sp., Erwinia sp., Bacillus sp., Streptomyces sp., Escherichia sp. (e.g., E. coli), and Klebsiella sp., among many others. The cells can be of any of several genera, including Saccharomyces (e.g., S. cerevisiae), Candida (e.g., C. utilis, C. parapsilosis, C. krusei, C. versatilis, C. lipolytica, C. zeylanoides, C. guilliermondii, C. albicans, and C. humicola), Pichia (e.g., P. farinosa and P. ohmeri), Torulopsis (e.g., T. candida, T. sphaerica, T. xylinus, T. famata, and T. versatilis), Debaryomyces (e.g., D. subglobosus, D. cantarellii, D. globosus, D. hansenii, and D. japonicus), Zygosaccharomyces (e.g., Z. rouxii and Z. bailii), Kluyveromyces (e.g., K. marxianus), Hansenula (e.g., H. anomala and H. jadinii), and Brettanomyces (e.g., B. lambicus and B. anomalus).

Promoters for use in E. coli include the T7, trp, or lambda promoters. A ribosome binding site and preferably a transcription termination signal are also provided. For expression of heterologous proteins in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli. Methods of transforming prokaryotes other than E. coli are well known. For example, methods of transforming Bacillus species and promoters that can be used to express proteins are taught in U.S. Pat. No. 6,255,076 and U.S. Pat. No. 6,770,475.

In yeast, convenient promoters include GAL1-10 (Johnson and Davies (1984) Mol. Cell. Biol. 4:1440-1448) ADH2 (Russell et al. (1983) J. Biol. Chem. 258:2674-2682), PHO5 (EMBO J. (1982) 6:675-680), and MFα (Herskowitz and Oshima (1982) in The Molecular Biology of the Yeast Saccharomyces (eds. Strathem, Jones, and Broach) Cold Spring Harbor Lab., Cold Spring Harbor, N.Y., pp. 181-209). Another suitable promoter for use in yeast is the ADH2/GAPDH hybrid promoter as described in Cousens et al., Gene 61:265-275 (1987). For filamentous fungi such as, for example, strains of the fungi Aspergillus (McKnight et al., U.S. Pat. No. 4,935,349), examples of useful promoters include those derived from Aspergillus nidulans glycolytic genes, such as the ADH3 promoter (McKnight et al., EMBO J. 4: 2093 2099 (1985)) and the tpiA promoter. An example of a suitable terminator is the ADH3 terminator (McKnight et al.).

In some embodiments, the polynucleotides are placed under the control of an inducible promoter, which is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, which allow one to control the timing of expression of the glycosyltransferase or enzyme involved in nucleotide sugar synthesis. For E. coli and other bacterial host cells, inducible promoters are known to those of skill in the art. These include, for example, the lac promoter. A particularly preferred inducible promoter for expression in prokaryotes is a dual promoter that includes a tac promoter component linked to a promoter component obtained from a gene or genes that encode enzymes involved in galactose metabolism (e.g., a promoter from a UDPgalactose 4-epimerase gene (galE)).

Inducible promoters for other organisms are also well known to those of skill in the art. These include, for example, the arabinose promoter, the lacZ promoter, the metallothionein promoter, and the heat shock promoter, as well as many others.

The construction of polynucleotide constructs generally requires the use of vectors able to replicate in bacteria. A plethora of kits are commercially available for the purification of plasmids from bacteria. For their proper use, follow the manufacturer's instructions (see, for example, EasyPrepJ, FlexiPrepJ, both from Pharmacia Biotech; StrataCleanJ, from Stratagene; and, QIAexpress Expression System, Qiagen). The isolated and purified plasmids can then be further manipulated to produce other plasmids, and used to transfect cells. Cloning in Streptomyces or Bacillus is also possible.

Selectable markers are often incorporated into the expression vectors used to construct the cells of the invention. These genes can encode a gene product, such as a protein, necessary for the survival or growth of transformed host cells grown in a selective culture medium. Host cells not transformed with the vector containing the selection gene will not survive in the culture medium. Typical selection genes encode proteins that confer resistance to antibiotics or other toxins, such as ampicillin, neomycin, kanamycin, chloramphenicol, or tetracycline. Alternatively, selectable markers may encode proteins that complement auxotrophic deficiencies or supply critical nutrients not available from complex media, e.g., the gene encoding D-alanine racemase for Bacilli. Often, the vector will have one selectable marker that is functional in, e.g., E. coli, or other cells in which the vector is replicated prior to being introduced into the target cell. A number of selectable markers are known to those of skill in the art and are described for instance in Sambrook et al., supra. A preferred selectable marker for use in bacterial cells is a kanamycin resistance marker (Vieira and Messing, Gene 19: 259 (1982)). Use of kanamycin selection is advantageous over, for example, ampicillin selection because ampicillin is quickly degraded by β-lactamase in culture medium, thus removing selective pressure and allowing the culture to become overgrown with cells that do not contain the vector.

Construction of suitable vectors containing one or more of the above listed components employs standard ligation techniques as described in the references cited above. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. Molecular cloning techniques to achieve these ends are known in the art. A wide variety of cloning and in vitro amplification methods suitable for the construction of recombinant nucleic acids are well-known to persons of skill.

A variety of common vectors suitable for constructing the recombinant cells of the invention are well known in the art. For cloning in bacteria, common vectors include pBR322 derived vectors such as pBLUESCRIP™, and λ-phage derived vectors. In yeast, vectors include Yeast Integrating plasmids (e.g., YIp5) and Yeast Replicating plasmids (the YRp series plasmids) and pGPD-2.

The methods for introducing the expression vectors into a chosen host cell are not particularly critical, and such methods are known to those of skill in the art. For example, the expression vectors can be introduced into prokaryotic cells, including E. coli, by calcium chloride transformation, and into eukaryotic cells by calcium phosphate treatment or electroporation. Other transformation methods are also suitable.

Methods for Producing Globosides

Production of Sialosyl galactosyl globosides is enhanced by manipulation of the host microorganism. For example, in E. coli, break down of sialic acid can be minimized by using a host strain that has diminished CMP-sialic acid synthase activity (NanA−). In E. coli, CMP− sialic acid synthase appears to be a catabolic enzyme. Diminishing the sialic acid degradative pathway in a host cell can be accomplished by disrupting the N-acetylneuraminate lyase gene (NanA, Accession number AE000402 region 70-963). Introduction of a sialyltransferase gene into these mutant strains results in a recombinant cell that is capable of producing large amounts of a sialylated product saccharide.

In some embodiments, the microorganisms are manipulated to enhance transport of an acceptor saccharide into the cell. Here, where lactose or globotriose is the acceptor saccharide, E. coli cells that express or overexpress the LacY permease can be used. Also in E. coli, when lactose is the acceptor saccharide or an intermediate in synthesizing the sialylated product, lactose breakdown can be minimized by using host cells that are LacZ−.

The invention provides methods in which the host cells are used to prepare globosides. As mentioned above, the culture medium may include lactose or globotriose as well as sialic acid or mannose and possibly other precursors to donor substrates or acceptor substrates.

Those of skill will recognize that culture medium for microorganisms can be e.g., rich mediums, such as Luria broth, animal free Luria broth, or Terrific broth or synthetic medium or semi-synthetic medium, such as M9 medium.

The methods of the invention can be used for producing globosides that are labeled with or enriched in radioisotopes; such oligosaccharides are extremely useful for fundamental biological or conformational analysis studies. The invention thus relates to a method for producing globosides that are labeled with at least one radioisotope. In these embodiments, the culture medium includes substrates labeled said radioisotope and/or in the presence of a said precursor labeled with said radioisotope. The radioisotopes are preferably chosen from the group composed of: 14C, 13C, 3H, 358, 32p, 33p.

The methods of the invention can also be used to activated oligosaccharides that may be used for the chemical synthesis of glycoconjugates or glycopolymers. The lactose acceptor can thus be modified such that glucose residue is replaced with an allyl group, said precursor now being allyl-13-D galactoside rather than lactose. For example, the double bond of the allyl group is chemically modified by addition, oxidation or ozonolysis reactions.

Methods and culture media for growth of microorganisms are well known to those of skill in the art. Culture can be conducted in, for example, aerated spinner or shaking culture, or, more preferably, in a fermentor.

The products produced by the above processes can be used without purification. However, it is usually preferred to recover the product. Standard, well known techniques for recovery of glycosylated saccharides such as thin or thick layer chromatography, column chromatography, ion exchange chromatography, or membrane filtration can be used. It is preferred to use membrane filtration, more preferably utilizing a reverse osmotic membrane, or one or more column chromatographic techniques for the recovery as is discussed hereinafter and in the literature cited herein.

Therapeutic and Other Uses

Globosides made according to the invention are useful in a wide range of therapeutic and diagnostic applications. They may be used, for example, as an agent for blocking cell surface receptors in the treatment of a host of diseases. As noted above, these oligosaccharides may be used for the chemical synthesis of glycoconjugates (e.g., glycolipids) or glycopolymers. The oligosaccharides or the glycoconjugates may be used, for example, as nutritional supplements, antibacterial agents, anti-metastatic agents and anti-inflammatory agents. The invention thus relates to the use of globosides according to the invention as a medicinal product in which the oligosaccharide or glycoconjugate is used to prepare a pharmaceutical composition. Methods for preparing pharmaceutical compositions are well known in the art.

In particular, it is envisionned to provide immunoadsorption therapies with the large scale preparation of globosides obtained according the method as defined above. Besides, globopentaose (Gb5) can be used in immunogenic composition for treating various cancers, in particular human embryonal carcinoma cells. Sialosyl galactosyl globoside can be used as an anti-infective agent.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. Citations are incorporated herein by reference.

### EXAMPLE 1

### Production of Globotriose

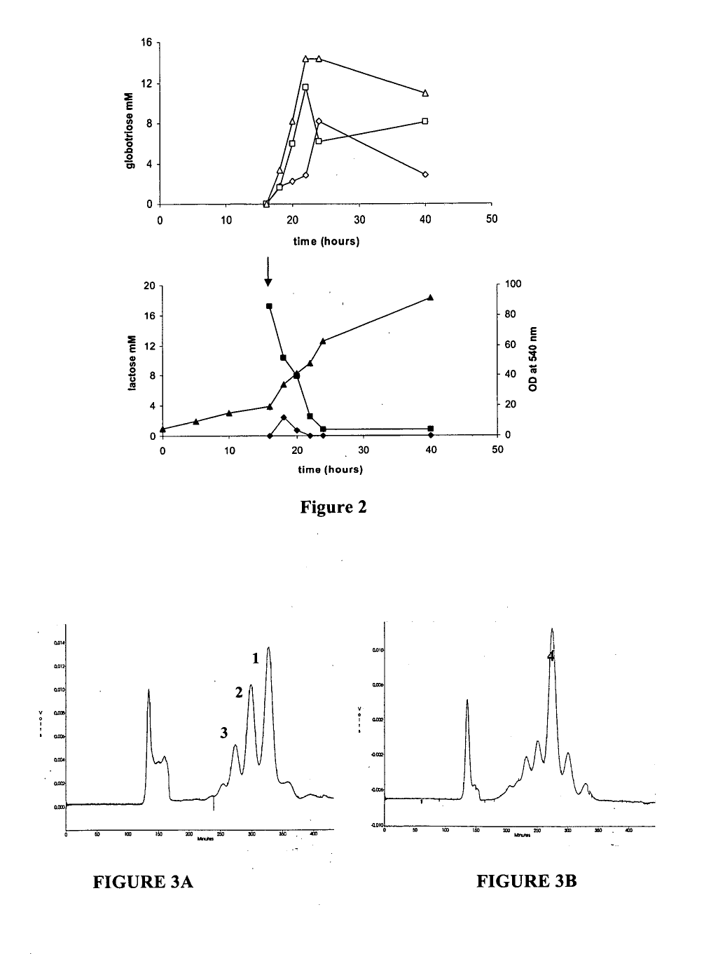

A strategy for producing globotriose (1) from exogenous lactose is described in FIG. 1. The host strain was an E. coli K12 strain JM107 derivative in which the melA gene for α-galactosidase activity (melibiase) had been inactivated [21]. The lgtC gene, encoding the α-1,4-galactosyltransferase, was amplified from the Neisseiria meningitidis L1 (126E) genome and cloned into a pBluescript plasmid. Sequencing revealed a frameshift in lgtC due to the deletion of a G in the polyG localised between the 157 bp and 170 bp positions. The reading frame was restored by site directed mutagenesis and the resulting plasmid (pBluescript-lgtC) was introduced in JM107melA⁻ to obtain the globotriose-producing strain TA19. Strain TA19 was cultured to high cell density with glycerol as the carbon and energy source (FIG. 2).

Lactose (14.6 mM) was added at the beginning of the fed-batch phase at the same time as the inducer IPTG. The production of globotriose (1) was followed by HPAEC-PAD analysis, which showed that lactose rapidly disappeared from the extracellular medium within 10 hours of culture. Lactose consumption correlated with both a small transient intracellular lactose accumulation and the appearance of a longer oligosaccharide, later identified as globotriose (1). Interestingly, the intracellular globotriose concentration rapidly plateaued after 3 hours of production whereas the extracellular globotriose concentration steadily increased until all the lactose had been consumed. At this point, a dramatic increase in the intracellular globotriose concentration occurred. Simultaneously a sharp decrease in the extracellular globotriose concentration was observed, suggesting that exogenous globotriose had been taken up by the cells after the complete exhaustion of lactose. Further culture of the cells resulted in a drop in the total globotriose concentration and HPAEC-PAD analysis indicated the simultaneous appearance of a series of longer oligosaccharides.

Cells were harvested after 40 hr of culture and the intracellular fraction was purified by charcoal adsorption and chromatographed on a Biogel P2 column (FIG. 3)

The separation profile confirmed the presence of a series of at least three compounds (1, 2 and 3) which were separated and further characterized. Mass spectral analysis of the purified compounds under the FAB+ mode showed the presence of peaks at m/z 505 and 427 (compound 1), 667 and 389 (compound 2), 829 and 851 (compound 3). These values represented the quasi molecular ions [M+H]⁺ and [M+Na]⁺ of a trihexose (compound 1), a tetrahexose (compound 2) and a pentahexose (compound 3). The identification of compound 1 as globotriose was confirmed by its ¹³C spectrum which showed one Cl of an α-Gal residue at δ=100,68 ppm and was in close agreement with previously reported assignments [10]. The ¹³C spectrum of compound 2 showed two Cl carbons of Gal residues at δ=101.47 ppm and δ=101.30 ppm, indicating that compound 2 could be identified as Galα-4Galα-4Galβ-4Glc.

### EXAMPLE 2

### Production of Globotetraose from Lactose

The system for globotriose production could be extended to the production of globotetraose by additionally expressing the lgtD and wbpP genes (FIG. 1). The lgtD gene encoding the β 1-3 N-acetylgalactosaminyltransferase activity was amplified from the Haemophilus influenza strain Rd DNA and was cloned upstream of lgtC in pBluescript-lgtC resulting in pBluescript-lgtDC. The globotetraose-producing strain TA11 was constructed by co-transforming the JM107melA⁻ strain with pBluescript-lgtDC and pBBR-wbpP which is a pBBR1-MCS2 derivative carrying the Pseudomonas aeruginosa wbpP gene for UDP-GlcNAc C4 epimerase [22].

The TA11 strain was cultured to a high cell density under the same conditions as for the production of globotriose by the TA19 strain. Globotetraose (4) production was monitored by determining the acid hydrolysable hexosamine concentration which increased linearly after the addition of lactose. After 40 hours of culture the final hexosamine concentrations in the intracellular and extracellular fractions were 2.0 mM and 4.5 mM respectively. HPAEC-PAD analysis revealed the presence of several oligosaccharides which could not be properly separated. Similarly, chromatography on Biogel P2 of the intracellular fraction showed a complex mixture of oligosaccharides (FIG. 3). The major compound (4) was further purified by reverse phase HPLC on a HP8NH2/25F column. The mass spectrum of purified compound 4 showed two peaks at m/z 708 and 730, corresponding to the quasi molecular ions [M+H]⁺ and [M+Na]⁺ derived from globotetraose. The ¹³C spectrum was in accordance with previously published data [15] [23] for globotetraose, and the chemical shifts 103.78 ppm, 53.15 ppm, 175.65 ppm and 22.77 ppm assigned to C-1, C-2 and carbons of N-acetyl group respectively clearly, indicated that a GalNAc residue was attached in β anomeric configuration to the globotriose.

To facilitate the identification of the minor compounds, the oligosaccharides of the intracellular fraction were reduced with ABEE. This technique provided an easy UV quantification of oligosaccharide derivatives as well as an amplified MS detection. Six compounds were separated by HPLC. Their molecular ions and characteristic fragments are indicated in Table 2.

TABLE 2
Mass spectroscopic characterization of oligosaccharides produced by the TA11 strain
after ABEE derivatization and separation by reverse-phase chromatography.

| | ESI positive-ionization data from purified ABEE oligosaccharides | | | | | |
|---|---|---|---|---|---|---|
| | (m/z) H⁺ and (Na⁺) | | | | | |
| | 5 | 6 | 7 | 8 | 9 | 10 |
| Molecular ions | 654 (676) | 816 (838) | 978 (1000) | 857 (879) | 1019 (1041) | 1181 (1203) |
| Fragment ions | | | | | | |
| Hex-ABEE | 330 | 330 | 330 | 330 | 330 | 330 |
| Hex₂-ABEE | 492 (514) | 492 (514) | 492 (514) | 492 (514) | 492 (514) | 492 |
| Hex₃-ABEE | | 654 (676) | 654 (676) | 654 | 654 (676) | 654 |
| Hex₄-ABEE | | | 816 (838) | | 816 (838) | |
| HexNAc | | | | 204 | 204 | 204 |

Compounds 5, 6 and 7 had molecular weights that corresponded to tri-, tetra and penta-hexose derivatives respectively. This strongly suggested that this series of compounds was identical to the series of galactooligosaccharides which had been previously identified in the culture of the TA19 strain. Compounds 8, 9, and 10 had the molecular weights of tetra, penta and hexa-hexose derivatives having an hexosamine residue. The formation of the ion fragment m/z 204 from compounds 8, 9 and 10 demonstrates that the hexosamine residue was always located at the non-reducing end of the oligosaccharides. This was confirmed by the formation of the ion fragment m/z 654 (corresponding to the [Hex₃ ABEE]⁺ group) from compound 8 and by the formation of the ion fragment m/z 816 (corresponding to the [Hex₄ ABEE]⁺ group) from compound 9. Assuming that the hexamine is a β1-3 linked GalNAc added onto globotriose and its mono and di-galactosylated derivatives, the three GalNAc containing oligosaccharides produced by the TA11 strain can thus be identified as

GalNAcβ-3Galα-4Galβ-4Glc (8),

GalNAcβ-3Galα-4Galα-4Galβ-4Glc (9) and

GalNAcβ-3Galα-4Galα-4Galα-4Galβ-4Glc (10)

### EXAMPLE 3

### Production of Globotetraose from Globotriose

The observation that extracellular globotriose was taken up by the cells after the complete exhaustion of lactose during the culture of the TA19 strain led us to investigate the possibility of using globotriose (1) as an exogenous acceptor for globotetraose synthesis as illustrated in FIG. 4.

The globotetraose-producing strain TA21 was constructed by co-transforming the JM107melA strain with pBBR-wbpP and pACT3-lgtD. The TA21 strain was cultured at high cell density and globotriose (1) (1.27 mM) was added instead of lactose as the acceptor. Analysis of oligosaccharide content in the intracellular and the extracellular fraction by thin layer chromatography indicated that globotriose (1) was rapidly internalized and entirely converted into globotetraose (4) within 6 hours of culture (FIG. 5).

Its final yield was estimated to be 1.25 mM by colorimetric quantification of acid-hydrolysable hexosamine. Since it was the only oligosaccharide remaining at the end of the culture, the globotetraose (4) was easily purified from the intracellular fraction by chromatography on Biogel P2 and its identity was confirmed by mass spectrometry.

### EXAMPLE 4

### Production of Globotriose without Polygalactosylated Side-Products

The polygalactosylated tetrasaccharide (Galα-4Galα-4Galβ-4Gal) and pentasaccharide (Galα-4Galα-4Galα-4Galβ-4Gal) were found in large amount in the cells of strain TA19 that were harvested after 40 hours of culture (FIG. 3). The formation of these side-products has two detrimental impacts on the process of globotriose production by the strain TA19. First it reduces the overall globotriose yield. Secondly it makes the globotriose purification more difficult to achieve.

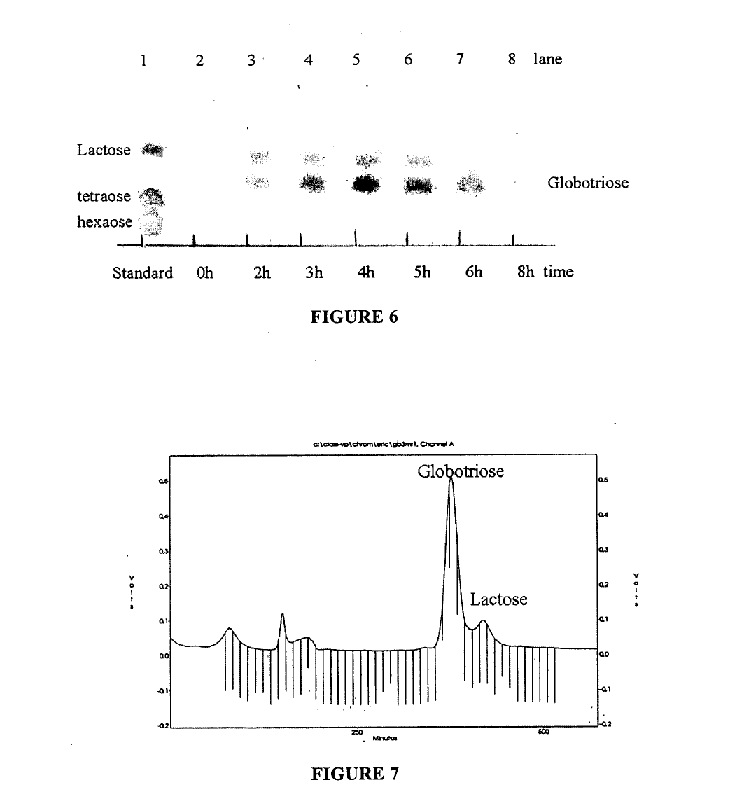

To improve the globotriose yield we have increased the concentration of lactose to 7.5 g.L−1 and carefully monitored the formation of oligosaccharide by TLC analysis. The FIG. 6 shows that globotriose was the only oligosaccharide produced by strain TA19 during the first 8 hours that followed the addition of lactose. After 8 hours, lactose was almost entirely consumed and the culture was stopped to prevent a further galactosylation of globotriose.

Globotriose was recovered from both the intracellular and the extracellular fraction of the 8 hours cultures of strain TA19 and purified by adsorption on activated charcoal. From a 7 liter culture of strain TA19, 35 g of charcoal purified globotriose fraction were obtained. Chromatography on Biogel P2 showed the presence of small amount of residual lactose but confirmed the absence of contaminating polygalactosylated compounds and the presence of globotriose as the major product (FIG. 7.)

### EXAMPLE 5

### Production of Globopentaose (Stage Specific Embryonic Antigen-3 SSEA-3)

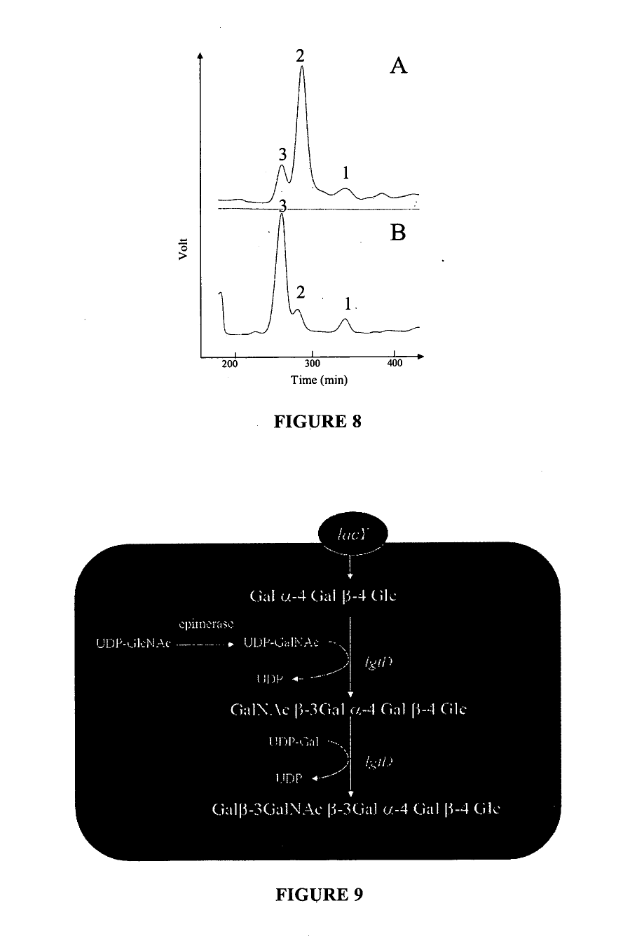

As described above in example 3, globotriose is rapidly converted into globotetraose by the strain TA21 and, after 6 hours of incubation, globotetraose was the major sugar recovered by chromatography on Biogel P2 from the intracellular oligosaccharide fraction of strain TA21 culture. However, the Biogel P2 chromatogram showed the presence of a small amount of an unidentified sugar (peak 3 FIG. 8A) which was larger than the globotetraose.

The strain TA21 was cultivated for a prolonged period of time and the analysis by chromatography on Biogel P2 showed that, after 20 hours of culture, the unidentified sugar has become the major compound detected in the intracellular fraction (FIG. 8B). Concurrently a decrease in the globotetraose concentration was also observed. The ESI⁺ mass spectrum of the peak 3 fraction showed the presence of a quasi molecular ions [M+Na]⁺ at m/z 892 which could originated from a pentasaccharide having 4 hexose residues and one N-acetylhexosamine residue. The determination of the monomeric sugar composition by HPAEC-PAD analysis after acid hydrolysis indicated that the pentasaccharide was made of Glc, Gal and GalNAc in a ratio of 1/3/1. This strongly suggests that the pentasaccharide was formed by the transfert of one Gal residue on a molecule of globotetraose. Compared with the NMR spectrum of the globotetraose, the ¹³C NMR spectrum of the pentasaccharide showed an additional signal at 105.6 ppm indicating that the third Gal residue was attached on the GalNAc with a P linkage. The linkage position on the GalNAc was determined by GC-MS analysis after methylation, acid-hydrolysis, reduction and acetylation of the pentasaccharide. The presence of fragment ions at m/z 261 and 161 derived from the GalNAc residue unambiguously demonstrates that the GalNAc was substituted on the 3 position.

These results clearly show that the pentasaccharide produced by strain TA21 after 20 hours of culture has the structure of the globopentaose: Galβ-3GalNAcβ-3Galα-4Galβ-4Gal. Since E. coli K12 is not known to have a β-3 Gal transferase activity, it is very likely that the conversion of globotriose into globotetraose has been catalysed by LgtD which is the only heterologous glycosyltransferase overexpressed in strain TA21. LgtD is thus a bifunctional glycosyltransferase having a β-3 GalNAc activity when globotriose is the acceptor and a β-3 Gal transferase activity when globotetraose is the acceptor. In addition, enzymatic assays with crude extracts from a strain overexpressing the lgtD gene indicated that the LgtD protein had both a galactosyl- and a N-acetylgalactosaminyl-transferase activity in presence of globotriose or globotetraose as acceptor. The maximum velocity rates of these two activities were in the same range whatever the acceptor and the sugar donor. However, large differences in the affinity of the enzyme for UDP-Gal or UDP-GalNAc were observed as a function of the acceptor used. When globotriose was the acceptor, the Km was 6 fold lower for UDP-GalNAc than for UDP-Gal indicating that lgtD act primarily as a GalNAc transferase converting globotriose into globotetraose. Replacement of globotriose by globotetraose as the acceptor resulted in 20 fold increase in the Km for UDP-GalNAc but on the contrary in a 3.5 fold decrease in the Km for UDP-Gal. In presence of globotetraose as the acceptor the enzyme, which had an 11 fold better affinity for UDP-Gal than for UDP-GalNAc, can thus be regarded as a galactosyltransferase which specifically direct the synthesis of globopentaose.

It has already been reported that some GalNAc transferases have a low Gal transferase activity but they normally have the same specificity for the acceptor whatever UDP-Gal or UDP GalNAc was used as sugar donor. On the contrary the acceptor specificities of the two glycosyltransferase activities of LgtD are very high because we never detected the formation of a tetrasaccharide different from globotetraose and of a pentasaccharide different from globopentaose. To our knowledge, the specific β-3gal transferase activity of LgtD with globotetraose as the acceptor has never been reported before.

The lgtD gene (HI1578) from Haemophilus influenzae strain rd can thus be advantageously used for the conversion of globotriose into globopentaose by using a metabolically engineered strain that is devoid of α and β galactosidase activity, and coexpresses the lgtD gene with a gene encoding a UDP-GlcNAc C4 epimerase activity as illustrated in FIG. 9.

### EXAMPLE 5

### Bis: Optimization of Globotetraose and Globopentaose Production

In order to optimize the globopentaose production, the wbpP gene was replaced by the C. jejuni gne gene, which has been shown to encode a more active UDP-GlcNAc C4 epimerase (Bernatchez et al., 2005), The gne gene was cloned by PCR from the genomic DNA of C. jejuni strain NCTC 11168 and both gne and lgtD were cloned together on the high copy number pBluescript plasmid to yield pBS-lgtD-gne.

TLC analysis of the intracellular oligosaccharide content of strain MR15 indicated that the globotetraose production rate was significantly improved when compared with strain TA21 with carried the plasmids pACT3-lgtD and pBBR-wbpP. Globotriose (3 g.l⁻¹) was entirely converted into glotetraose within 4 hours of incubation by strain MR15, whereas it took 6 hours for the strain TA21 to convert only 1 g.l¹ of globotriose. After purification by charcoal adsorption and size exclusion chromatography the yield of pure globopentaose was 1.29 g from a one liter culture.

### EXAMPLE 6

### Production of Globo-H Hexasaccharide

We have already shown that fucosylated oligosaccharide can be produced in living E. coli cells that have been metabolically engineered to overexpress the genes involved in GDP-Fucose biosynthesis and the appropriate glycosyltransferase genes [26]. It has also been reported that the Helicobacter pylori gene futC which encodes an α-2 fucosyltransferase is functionally expressed in E. coli [25]. Therefore, we provide a new method for producing the Globo-H hexasaccharide (Fucα-2Galβ-3GalNAcβ-3Galα-4Galβ-4Gal) from globotriose using a strain that overexpresses the lgtD and futC genes as illustrated in FIG. 10. In one embodiment, to prevent a possible fucosylation of the globotriose, a mutant manA⁻ devoid of phophomannose isomerase activity can be used. This mutant will be unable to synthesize GDP-Fuc, unless mannose is exogenously added in the medium and taken up and phosphorylated in Man-6-P by the mannose permease encoded by the manXXZ genes. By adding the mannose after the entire conversion of globotriose into globopentaose, the fucosylation of globotriose will be impossible and all the globotriose will be converted into Globo-H oligosaccharide. In another preferred embodiment, the strain MR20 was constructed by transforming the host strain DM with the three plasmids pBS-lgtD-gne, pBBRGAB and pWKS-lgtC. The host strain DM was a melA null derivative of strain DC (Dumon et al., 2006). The plasmid pBBRGAB contained the four genes gmd, wcaG, manC manB required for the synthesis of GDP-Fuc (Dumon et al., 2006). The plasmid pWKS-lgtC was constructed by cloning the futC gene from pEXT20futC (Drouillard et al., 2006) into the KpnI SalI sites of pWKS130 yielding pWKS-futC. The strain MR20, was cultivated in presence of 3 g.l⁻¹ of globotriose. TLC analysis shows that, after 23 h of incubation, globotriose was entirely converted into globotetraose (3) and a compound (5) that migrates as an hexaose (FIG. 11). Prolonged incubation resulted in the almost complete conversion of Globotetraose into compound (5). Compound (5) was purified on activated charcoal and by size exclusion chromatography on Biogel P2 with a yield of 1.59 gram from a one liter culture and its identification as globo-H sugar was confirmed by mass spectrometry and NMR analysis. During the culture there was no accumulation of Globopentaose indicating that the fucosyltransferase was very active on globopentaose. On the other hand there was no significant formation of a compound that could migrate as a fucosylated globotriose suggesting that Globotriose was not a good substrate for the fucosyltransferase.

### EXAMPLE 7

### Production of Sialosyl Galactosyl Globoside (SGG) Hexasaccharide (Stage Specific Embryonic Antigen-4, SSEA-4)

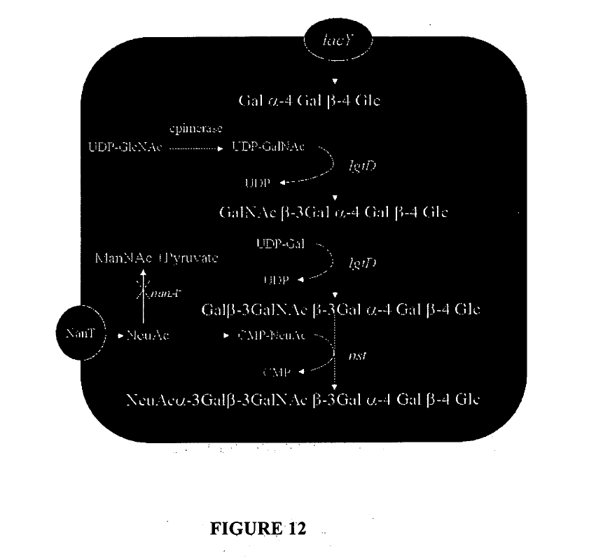

It has already been reported that 3′sialyllactose (NeuAcα-3Galβ-4Glc) can be produced from exogenous lactose and sialic acid (NeuAc) by metabolically engineered living E. coli cells that overexpressed heterologous gene nst for α-3 sialyltransferase and for the CMP-NeuAc synthase (17). We have combined this sialylation system with the system of globopentaose synthesis as described in example 5 to produce the SGG hexasaccharide (NeuAcα-3Galβ-3GalNAcβ-3Galα-4Galβ-4Gal) from exogenous globotriose and NeuAc as shown in FIG. 12. To prevent a possible sialylation of the globotriose, NeuAc is added after the entire conversion of globotriose into globotetraose.

### EXAMPLE 8

### Production of the Terminal Epitope of the SSEA-3, Globo-H Antigen and SSEA-4 Using Gal as Acceptor

A galK mutant lacking galactokinase activity can use galactose as acceptor for the synthesis of oligosaccharide with a terminal gal residue. The strain MR16 was constructed by transforming the lacZ⁻ galK⁻ host strain host GLK with the pBS-lgtD-gne plasmid. Culture of strain MR16 in presence of galactose resulted in the formation of the terminal trisaccharide structure of the SSEA-3 antigen (Galβ-3GalNAcβ-3Gal) with a transient accumulation of the disaccharide intermediate GalNAcβ-3Gal. These results demonstrate that the LgtD act as GalNAc transferase in presence of Gal and as a Gal transferase in presence of GalNAcβ-3Gal.

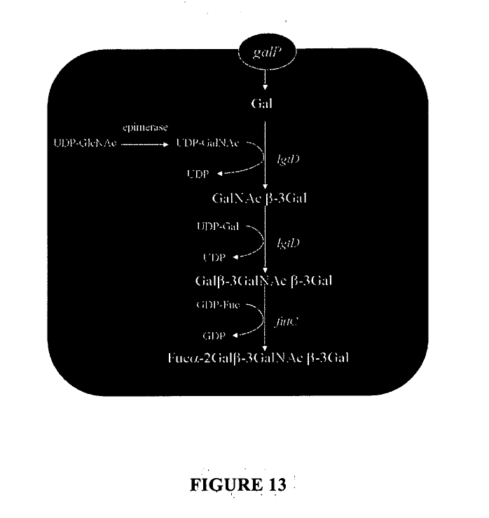

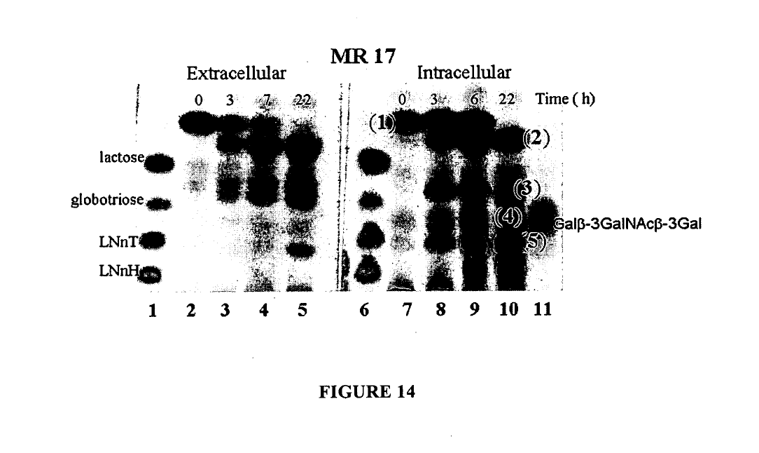

This method can be extended to the production of the terminal tetrasaccharide structure of the Globo-H antigen (Fucα-2Galβ-3GalNAcβ-3Gal) as described in FIG. 13. Similarly, the system for the synthesis of the sialosyl galactosyl globoside (SGG) hexasaccharide described in example 7 can be adapted to the synthesis of the terminal tetrasaccharide epitope of the SSEA-4 antigen (NeuAcα-3Galβ-3GalNAcβ-3Gal) by using Gal as the acceptor.

### EXAMPLE 9

### Production of Globo-H Hexasaccharide with a Terminal Propargyl Group

The strain MR20 was cultivated as in example 6 except that propargyl-β-globotrioside (1 g.l⁻) was used as acceptor instead of globotriose. At the end of the culture the propargyl-β-globotrioside was entirely consumed and the main oligosaccharide product was a compound that migrated in TLC as an hexasaccharide and which was purified by charcoal adsorption and size exclusion chromatography on Biogel P2 with a yield of 0.53 gram from a one liter culture. This compound was identified as Fucα-2Galβ-3GalNAcβ-3Galα-4Galβ-4Galβ-propargyl by mass spectrometry (EI-MS: m/z [M+Na]⁺=1076,) and NMR analysis. Selected ¹H signals

¹H-NMR (D₂O, 293K): δ (ppm)=5.24 (H-1″″, d, J=2 Hz, 1H), 4.90 (H-1″, d, J=2 Hz, 1H), 4.68, 4.63, 4.55, 4.52 (H-1, H-1′, H-1′″, H-1″″, 4d, J=7.8 Hz, 4H), 4.51 (CH₂ Prop, s, 2H), 3.35 (H-2β, m, J=7.8 Hz, 1H), 2.04 (COCH₃, s, 3H), 1.23 (H-6′″″, d, J=6.8 Hz, 3H). ¹³C NMR (D₂O, 303K): δ (ppm)=175.48 (CO), 105.16, 104.49, 103.25, 101.64, 101.57 (C-1, C-1′, C-1″, C-1′″, C-1″″), 100.45 (C-1′″″), 57.78 (CH₂Prop), 52.84 (C-2′″), 23.46 (CH₃), 16.50 (C-6″″″).

### EXAMPLE 10

### Production of Globo-H Tetrasaccharide with a Terminal Propargyl Group

The strain MR17 was cultivated as in example 8 except that propargyl-β-galactose (3 g.l⁻) was used as acceptor instead of galactose. The major oligosaccharide recovered at the end of the culture migrated as a tetrasaccharide in TLC and was purified with a yield of 1.65 g from a one liter culture. This compound was identified as Fucα-2Galβ-3GalNAcβ-3Galβ-propargyl by mass spectrometry (EI-MS: m/z [M+Na]⁺=752) and NMR analysis. Selected ¹H signals ¹H-NMR (D₂O, 293K): δ (ppm)=5.24 (H-1′″, d, J=2 Hz, 1H), 4.62, 4.56 (H-1, H-1′, H-1″, 2d, J=7.8 Hz, 3H), 4.48 (CH₂ Prop, s, 2H), 4.25 (H-5′″, q, J=6.8 Hz, 1H), 2.04 (COCH₃, s, 3H), 1.23 (H-6′″, d, J=6.8 Hz, 3H). ¹³C NMR (D₂O, 303K): δ (ppm)=175.52 (CO), 105.25, 103.21, 102.58 (C-1, C-1′, C-1″), 100.50 (C-1′″), 57.82 (CH₂Prop), 52.89 (C-2′), 23.54 (CH₃), 16.50 (C-6′″).

### REFERENCES

[1] A. A. Lindberg, J. E. Brown, N. Stromberg, M. Westling-Ryd, J. E. Schultz, and K. A. Karlsson, Identification of the carbohydrate receptor for Shiga toxin produced by Shigella dysenteriae type 1, J Biol Chem 262 (1987) 1779-1785.
[2] K. E. Brown, S. M. Anderson, and N. S. Young, Erythrocyte P antigen: cellular receptor for B19 parvovirus, Science 262 (1993) 114-117.
[3] P. R. Chipman, M. Agbandje-McKenna, S. Kajigaya, K. E. Brown, N. S. Young, T. S. Baker, and M. G. Rossmann, Cryo-electron microscopy studies of empty capsids of human parvovirus B19 complexed with its cellular receptor, Proc Natl Acad Sci USA 93 (1996) 7502-7506.
[4] J. A. Roberts, B. I. Marklund, D. Ilver, D. Haslam, M. B. Kaack, G. Baskin, M. Louis, R. Mollby, J. Winberg, and S. Normark, The Gal(alpha 1-4)Gal-specific tip adhesin of Escherichia coli P-fimbriae is needed for pyelonephritis to occur in the normal urinary tract, Proc Natl Acad Sci USA 91 (1994) 11889-11893.
[5] R. J. Scholten, B. Kuipers, H. A. Valkenburg, J. Dankert, W. D. Zollinger, and J. T. Poolman, Lipo-oligosaccharide immunotyping of Neisseria meningitidis by a whole-cell ELISA with monoclonal antibodies, J Med Microbiol 41 (1994) 236-243.
[6] E. C. Gotschlich, Genetic locus for the biosynthesis of the variable portion of Neisseria gonorrhoeae lipooligosaccharide, J Exp Med 180 (1994) 2181-2190.
[7] M. P. Jennings, D. W. Hood, I. R. Peak, M. Virji, and E. R. Moxon, Molecular analysis of a locus for the biosynthesis and phase-variable expression of the lacto-N-neotetraose terminal lipopolysaccharide structure in Neisseria meningitidis, Mol Microbiol 18 (1995) 729-740.
[8] W. W. Wakarchuk, A. Cunningham, D. C. Watson, and N. M. Young, Role of paired basic residues in the expression of active recombinant galactosyltransferases from the bacterial pathogen Neisseria meningitidis, Protein Eng 11 (1998) 295-302.
[9] K. F. Johnson, Synthesis of oligosaccharides by bacterial enzymes, Glycoconj J 16 (1999) 141-146.
[10] J. Zhang, P. Kowal, J. Fang, P. Andreana, and P. G. Wang, Efficient chemoenzymatic synthesis of globotriose and its derivatives with a recombinant alpha-(1-->4)-galactosyltransferase, Carbohydr Res 337 (2002) 969-976.
[11] S. Koizumi, T. Endo, K. Tabata, and A. Ozaki, Large-scale production of UDP-galactose and globotriose by coupling metabolically engineered bacteria, Nat Biotechnol 16 (1998) 847-850.
[12] J. Zhang, P. Kowal, X. Chen, and P. G. Wang, Large-scale synthesis of globotriose derivatives through recombinant E. coli, Org Biomol Chem 1 (2003) 3048-3053.
[13] A. Risberg, G. Alvelius, and E. K. Schweda, Structural analysis of the lipopolysaccharide oligosaccharide epitopes expressed by Haemophilus influenzae strain RM. 118-26, Eur J Biochem 265 (1999) 1067-1074.
[14] D. W. Hood, A. D. Cox, W. W. Wakarchuk, M. Schur, E. K. Schweda, S. L. Walsh, M. E. Deadman, A. Martin, E. R. Moxon, and J. C. Richards, Genetic basis for expression of the major globotetraose-containing lipopolysaccharide from H. influenzae strain Rd (RM118), Glycobiology 11 (2001) 957-967.
[15] J. Shao, J. Zhang, P. Kowal, and P. G. Wang, Donor substrate regeneration for efficient synthesis of globotetraose and isoglobotetraose, Appl Environ Microbiol 68 (2002) 5634-5640.
[16] J. Shao, J. Zhang, P. Kowal, Y. Lu, and P. G. Wang, Efficient synthesis of globoside and isogloboside tetrasaccharides by using beta(1-->3) N-acetylgalactosaminyltransferase/UDP-N-acetylglucosamine C4 epimerase fusion protein, Chem Commun (Camb) (2003) 1422-1423.
[17] B. Priem, M. Gilbert, W. W. Wakarchuk, A. Heyraud, and E. Samain, A new fermentation process allows large-scale production of human milk oligosaccharides by metabolically engineered bacteria, Glycobiology 12 (2002) 235-240.
[18] D. M. Dykxhoorn, R. St Pierre, and T. Linn, A set of compatible tac promoter expression vectors, Gene 177 (1996) 133-136.
[19] D. T. Li, and G. R. Her, Structural analysis of chromophore-labeled disaccharides and oligosaccharides by electrospray ionization mass spectrometry and high-performance liquid chromatography/electrospray ionization mass spectrometry, J Mass Spectrom 33 (1998) 644-652.
[20] S. Suzuki, K. Kakehi, and S. Honda, Comparison of the sensitivities of various derivatives of oligosaccharides in LC/MS with fast atom bombardment and electrospray ionization interfaces, Anal Chem 68 (1996) 2073-2083.
[21] E. Bettler, A. Imberty, B. Priem, V. Chazalet, A. Heyraud, D. H. Joziasse, and R. A. Geremia, Production of recombinant xenotransplantation antigen in Escherichia coli, Biochem Biophys Res Commun 302 (2003) 620-624.
[22] T. Antoine, B. Priem, A. Heyraud, L. Greffe, M. Gilbert, W. W. Wakarchuk, J. S. Lam, and E. Samain, Large-scale in vivo synthesis of the carbohydrate moieties of gangliosides GM1 and GM2 by metabolically engineered Escherichia coli, Chembiochem 4 (2003) 406-412.
[23] U. Nilsson, A. K. Ray, and G. Magnusson, Synthesis of the globotetraose tetrasaccharide and terminal tri- and di-saccharide fragments, Carbohydr Res 252 (1994) 117-136.
[24] G. Cornelis, R. K. Luke, and M. H. Richmond, Fermentation of raffinose by lactose-fermenting strains of Yersinia enterocolitica and by sucrose-fermenting strains of Escherichia coli, J Clin Microbiol 7 (1978) 180-183.
[25] Wang, G., P. G. Boulton, et al. (1999). “Novel Helicobacter pylori alpha1,2-fucosyltransferase, a key enzyme in the synthesis of Lewis antigens.” Microbiology 145 (Pt 11): 3245-53.
[26] Dumon, C., B. Priem, et al. (2001). “In vivo fucosylation of lacto-N-neotetraose and lacto-N-neohexaose by heterologous expression of Helicobacter pylori alpha-1,3 fucosyltransferase in engineered Escherichia coli.” Glycoconi J 18(6): 465-74.
[27] Wenk, J., P. W. Andrews, et al. (1994). “Glycolipids of germ cell tumors: extended globo-series glycolipids are a hallmark of human embryonal carcinoma cells.” Int J Cancer 58(1): 108-15.
[28] Slovin, S. F., G. Ragupathi, et al. (1999). “Carbohydrate vaccines in cancer: immunogenicity of a fully synthetic globo H hexasaccharide conjugate in man.” Proc Natl Acad Sci USA 96(10): 5710-5.
[29] Krupnick, J. G., I. Damjanov, et al. (1994). “Globo-series carbohydrate antigens are expressed in different forms on human and murine teratocarcinoma-derived cells.” Int J Cancer 59(5): 692-8.
[30] Stapleton, A. E., M. R. Stroud, et al. (1998). “The globoseries glycosphingolipid sialosyl galactosyl globoside is found in urinary tract tissues and is a preferred binding receptor In vitro for uropathogenic Escherichia coli expressing pap-encoded adhesins.” Infect Immun 66(8): 3856-61
[31] Ito, A., S. Saito, et al. (2001). “Monoclonal antibody (5F3) defining renal cell carcinoma-associated antigen disialosyl globopentaosylceramide (V3NeuAcIV6NeuAcGb5), and distribution pattern of the antigen in tumor and normal tissues.” Glycoconi J 18(6): 475-85.

1. A method for producing an oligosaccharide comprising the galabiose motif (Galα-4Gal), referred as globosides, the method comprising culturing a first microorganism which is LacY+ (β-galactoside permease), LacZ− (β galactosidase), and MelA− (α-galactosidase) in a culture medium comprising lactose, wherein said first microorganism comprises a heterologous lgtC gene encoding α-1,4-Gal transferase which transfers a galactose moiety from UDP-Gal to the lactose to form globotriose (Galα-4Galβ-4Glc) and wherein lactose is in excess in the culture medium.

2. The method of claim 1, wherein the culture is terminated before the exhaustion of lactose and globotriose is extracted from the culture medium.

3. The method of claim 1, wherein the LgtC gene is from Neisseria meningititis.

4. A microorganism comprising a heterologous lgtC gene encoding α-1,4-Gal transferase and which is LacY+ (β-galactoside permease), LacZ− (β galactosidase), and MelA− (α-galactosidase).

5. A cell culture medium comprising lactose in excess and the microorganism of claim 4.

6. A commercial scale composition comprising at least 80% by weight globotriose obtained by the method of claim 2.

7. The method of claim 1, wherein said microorganism further comprises a heterologous lgtD gene encoding β-3 GalNAc transferase which transfers a GalNAc moiety from UDP-GalNAc to the globotriose to form globotetraose (GalNAcβ-3Galα-4Galβ-4Glc).

8. The method of claim 7, wherein said microorganism further comprises a wbpP encoding for UDP-GlcNAc-C4 epimerase, such as the Pseudomonas aeruginosa wbpP gene; or a gne gene encoding for a UDP-glucose 4-epimerase, such as the gne gene of C. jejuni strain NCTC 11168 of SEQ ID No 9.

9. The microorganism of claim 4 further comprising a heterologous lgtD gene encoding β-3 GalNAc transferase and a wbpP gene encoding for UDP-GlcNAc-C4 epimerase or a gne gene encoding for a UDP-glucose 4-epimerase, such as the gne gene of C. jejuni strain NCTC 11168 of SEQ ID No 9.

10. The cell culture medium comprising lactose in excess and the microorganism of claim 9.

11. The method of claim 2 further comprising providing globotriose to the culture medium of a second microorganism, wherein said second microorganism comprises a heterologous a lgtD gene encoding β-3 GalNAc transferase which transfers a GalNAc moiety from UDP-GalNAc to globotriose to form globotetraose (GalNAcβ-3Galα-4Galβ-4Glc).

12. The method of claim 11, wherein said second microorganism is LacY+, LacZ−, melA− and comprises a wbpP gene encoding for UDP-GlcNAc-C4 epimerase, such as the Pseudomonas aeruginosa wbpP gene or a gne gene encoding for a UDP-glucose 4-epimerase, such as the gne gene of C. jejuni strain NCTC 11168 of SEQ ID No 9.

13. The method of claim 11, wherein said first and second microorganisms are recombinant E. coli strains.

14. The method of claim 11, wherein glycerol is used in the media of said first and second microorganisms as carbon and energy source.

15. A microorganism which is LacY+, LacZ−, melA− and comprises a heterologous a lgtD gene encoding β-3 GalNAc transferase, and a wbpP gene encoding for UDP-GlcNAc-C4 epimerase, such as the Pseudomonas aeruginosa wbpP gene or a gne gene encoding for a UDP-glucose 4-epimerase, such as the gne gene of C. jejuni strain NCTC 11168 of SEQ ID No 9.

16. A set of two separate microorganisms, comprising said first microorganism of claim 4 and said second microorganism which is LacY+, LacZ−, melA− and comprises a heterologous a lgtD gene encoding β-3 GalNAc transferase, and either a wbpP gene encoding for UDP-GlcNAc-C4 epimerase or a gne gene encoding for a UDP-glucose 4-epimerase, such as the gne gene of Campylobacter jejuni strain NCTC 11168 of SEQ ID No 9.

17. A cell culture medium comprising globotriose and a microorganism of claim 15.

18. The method of claim 11 further comprising extending the culture to allow said lgtD gene encoding β-3 GalNAc transferase to transfer a galactose moiety from UDP-Gal to globotetraose to form globopentaose (Galβ-3GalNAcβ-3Galα-4Galβ-4Gal).

19. A method for catalyzing the transfer of a galactose moiety from UDP-Gal to globotetraose to form globopentaose (β-3 Gal transferase activity) comprising catalyzing the transfer with a lgtD gene encoding β-3 GalNAc transferase, in particular the lgtD gene from Haemophilus influenzae of SEQ ID No 3.

20. The method of claim 18, wherein said second microorganism further comprises a heterologous futC gene encoding an α-2 fucosyltranferase to transfer a fucose moiety from GDP-Fuc to globopentaose to form Globo-H hexasaccharide (Fucα-2Galβ-3GalNAcβ-3Galα-4Galβ-4Gal).

21. The method of claim 20 wherein mannose is added in the medium after the entire conversion of globotriose into globopentaose.

22. The microorganism of claim 20 which is LacY+, (optionally MelA−, manXXZ+), manA⁻ and which comprises a heterologous a lgtD gene (β-3 GalNAc transferase), a heterologous wbpP gene (UDP-GlcNAc-C4 epimerase), such as the Pseudomonas aeruginosa wbpP gene or a gne gene encoding for a UDP-glucose 4-epimerase, such as the gne gene of C. Campylobacter jejuni strain NCTC 11168 of SEQ ID No 9 and a heterologous futC gene (α-2 fucosyltranferase), such as the Helicobacter pylori gene futC of SEQ ID No 5.

23. A set of two separate microorganisms, comprising said first microorganism comprising a heterologous lgtC gene encoding α-1,4-Gal transferase and which is LacY+, LacZ−, and MelA− and said second microorganism as defined in claim 22.

24. The method of claim 18, wherein said second microorganism further comprises a gene encoding the CMP-NeuAc synthase, such as a gene encoding the CMP-NeuAc synthase from N. meningitidis, and a heterologous gene encoding α-3 sialyltransferase, such as the α-3 sialyltransferase gene from N. meningitidis of SEQ ID No 7, which catalyzes the transfer of a sialyl moiety from an activated sialic acid molecule to globopentaose to form sialosyl galactosyl globoside (SGG) hexasaccharide (NeuAcα-3Galβ-3GalNAcβ-3Galα-4Galβ-4Gal).

25. The microorganism as defined in claim 24 which is LacY+, MelA−, nanT+, nanA⁻ and which comprises a heterologous a lgtD gene (β-3 GalNAc transferase), a heterologous wbpP gene (UDP-GlcNAc-C4 epimerase), such as the Pseudomonas aeruginosa wbpP gene and a heterologous gene for α-3 sialyltransferase, such as the gene from N. meningitidis of SEQ ID No 7.

26. A cell culture comprising the microorganism as defined in claim 25 and sialic acid.

27. A set of two separate microorganisms, comprising said first microorganism comprising a heterologous lgtC gene encoding α-1,4-Gal transferase and which is LacY+, LacZ−, and MelA− and said second microorganism as defined in claim 25.

28. A method for producing an oligosaccharide comprising the galabiose motif (Galα-4Gal), referred as globosides, selected the group consisting of globotetraose, globopentaose, and galactosyl-globosides including globo-H hexasaccharide, sialosyl galactosyl globoside (SGG) hexasaccharide, comprising the step consisting of culturing a microorganism as defined in claim 15 in a medium comprising globotriose.

29. A culture medium comprising globotriose at a concentration of 1 to 10 g/L.

30. The method of claim 2 for preparation of a culture medium comprising globotriose at a concentration of 1 to 10 g/L.

31. A commercial scale composition comprising one or more globoside selected from the group consisting of globotriose, globotetraose, globopentaose, and galactosyl-globosides including globo-H hexasaccharide, and sialosyl galactosyl globoside (SGG) hexasaccharide.

32. A method of making the commercial scale composition of claim 31 for the preparation of a nutritional supplement, comprising adding the commercial scale composition to a carrier.

33. The method of making the commercial scale composition of claim 31, wherein the composition is an antibacterial agent, anti-metastatic agent, anti-inflammatory agent, immunogenic composition such as for treating cancers in particular human embryonal carcinoma and for immunoadsorption therapies.

34. A method for producing an oligosaccharide comprising a galabiose motif (Galα-4Gal), the method comprising culturing a first microorganism in a culture medium comprising lactose, wherein said microorganism comprises a heterologous gene encoding α-1,4-Gal transferase which transfers a galactose moiety from UDP-Gal to the lactose to form globotriose (Galα-4Galβ-4Glc) and wherein said lactose is in excess in the culture medium.

35. The method of claim 34, wherein the culture is terminated before the exhaustion of lactose and said globotriose is extracted from the culture medium.

36. The method of claim 34, wherein said α-1,4-Gal transferase is an LgtC gene from Neisseria meningititis.

37. The method of claim 34, wherein said microorganism encodes a β-galactoside permease, lacks a functional β galactosidase gene, and lacks a functional α-galactosidase gene.

38. The method of claim 37, wherein the microorganism is an E. coli which is LacY+ (β-galactoside permease), LacZ− (β galactosidase), and MelA− (α-galactosidase).

39. The method of claim 34, wherein said microorganism further comprises a heterologous gene encoding β-3 GalNAc transferase, such as the LgtD gene from Neisseria meningititis, which transfers a GalNAc moiety from UDP-GalNAc to the globotriose to form globotetraose (GalNAcβ-3Galα-4Galβ-4Glc).

40. The method of claim 39, wherein said microorganism further comprises a gene encoding for UDP-GlcNAc-C4 epimerase or a UDP-glucose 4-epimerase.

41. The method of claim 39, wherein said gene encoding for UDP-GlcNAc-C4 epimerase is a Pseudomonas aeruginosa wbpP gene or a gne gene encoding for a UDP-glucose 4-epimerase, such as the gne gene of C. jejuni strain NCTC 11168 of SEQ ID No 9.

42. The method of claim 34 further comprising the step of providing globotriose to the culture medium of a second microorganism, wherein said second microorganism comprises a heterologous a gene encoding β-3 GalNAc transferase which transfers a GalNAc moiety from UDP-GalNAc to globotriose to form globotetraose (GalNAcβ-3Galα-4Galβ-4Glc).

43. The method of claim 42, wherein said β,3-GalNAc transferase is an LgtD gene from Neisseria meningititis.

44. The method of claim 42, wherein said second microorganism encodes β-galactoside permease, lacks a functional β galactosidase gene, and lacks a functional α-galactosidase gene.

45. The method of claim 44, wherein said second microorganism is an E. coli which is LacY+ (β-galactoside permease), LacZ− (β galactosidase), and MelA− (α-galactosidase) and comprises a gene encoding for UDP-GlcNAc-C4 epimerase.

46. The method of claim 45, wherein said gene encoding for UDP-GlcNAc-C4 epimerase is a Pseudomonas aeruginosa wbpP gene or a gne gene encoding for a UDP-glucose 4-epimerase, such as the gne gene of C. Campylobacter jejuni strain NCTC 11168 of SEQ ID No 9.

47. The method of claim 43, further comprising extending the culture to allow said lgtD gene encoding β-3 GalNAc transferase to transfer a galactose moiety from UDP-Gal to globotetraose to form globopentaose (Galβ-3GalNAcβ-3Galα-4Galβ-4Gal).

48. The method of claim 47, wherein said second microorganism further comprises a heterologous gene encoding an α-2 fucosyltranferase to transfer a fucose moiety from GDP-Fuc to globopentaose to form Globo-H hexasaccharide (Fucα-2Galβ-3GalNAcβ-3Galα-4Galβ-4Gal).

49. The method of claim 48, wherein said α-2 fucosyltranferase is a futC gene as shown in SEQ ID NO:5.

50. The method of claim 47, wherein said second microorganism further comprises a gene encoding a CMP-NeuAc synthase, and a heterologous gene encoding α-3 sialyltransferase, which catalyzes the transfer of a sialyl moiety from an activated sialic acid molecule to globopentaose to form sialosyl galactosyl globoside (SGG) hexasaccharide (NeuAcα-3Galβ-3GalNAcβ-3Galα-4Galβ-4Gal).

51. The method of claim 50, wherein said CMP-NeuAc synthase is from N. meningitidis, and said α-3 sialyltransferase comprises the sequence of SEQ ID NO:7.

52. A method of catalyzing the transfer of a galactose moiety from UDP-Gal to an acceptor bearing the terminal non reducing structure GalNAcβ-3-R to form the Galβ-3GalNAcβ-3-R structure, comprising catalyzing the transfer with the glycosyltransferase encoded by the lgtD gene from Haemophilus influenzae of SEQ ID NO:3 or a sequence having at least 80% identity thereof, as a β1,3 galactosyl transferase
wherein R is selected from the group consisting of galactose, β galactosides such as allyl-β-galactoside or propargyl-β-galactoside, α galactosides, globotriose, β globotrioside such as allyl-β-globotrioside or propargyl-β-globotrioside, α globotrioside, and galactose-X; and
wherein X is a reactive group allowing the covalent coupling with an other molecule, including amino, azide and nitrophenyl groups.

53. The method of claim 52, to produce an oligosaccharide selected from Galβ-3GalNAcβ-3Gal, Galβ-3GalNAcβ-3Galα-X, Galβ-3GalNAcβ-3Galβ-X, Galβ-3GalNAcβ-3Galβ-allyl, Galβ-3GalNAcβ-3Galβ-propragyl, Galβ-3GalNAcβ-3Galα-4Galβ-4Gal (Globopentaose), Galβ-3GalNAcβ-3Galβ-4Galβ-4Galα-X, Galβ-3GalNAcβ-3Galα-4Galβ-4Galβ-X, Galβ-3GalNAcβ-3Galα-4Galβ-4Galβ-allyl, and Galβ-3GalNAcβ-3Galα-4Galβ-4Galβ-propargyl.

54. A method of producing an oligosaccharide selected from Galβ-3GalNAcβ-3Gal, Galβ-3GalNAcβ-3Galα-X, Galβ-3GalNAcβ-3Galβ-X, Galβ-3GalNAcβ-3Galβ-allyl, Galβ-3GalNAcβ-3Galβ-propragyl, Galβ-3GalNAcβ-3Galα-4Galβ-4Gal (Globopentaose), Galβ-3GalNAcβ-3Galα-4Galβ-4Galα-X, Galβ-3GalNAcβ-3Galα-4Galβ-4Galβ-X, Galβ-3GalNAcβ-3Galα-4Galβ-4Galβ-allyl, and Galβ-3GalNAcβ-3Galα-4Galβ-4Galβ-propargyl, wherein said oligosaccharide is produced by a microorganism comprising an heterologous lgtD gene from Haemophilus influenzae of SEQ ID NO:3 or a sequence having at least 80% identity thereof.

55. A method of transferring a GalNAc residue to galactose to form GalNAcβ-3Gal and to produce oligosacharrides comprising GalNAcβ-3Gal, comprising a lgtD gene encoding a GalNAc transferase, in particular the lgtD from H. influenzae (SEQ ID NO:3).

56. The method of claim 1, comprising a lgtD gene encoding a GalNAc transferase, in particular the lgtD from H. influenzae (SEQ ID No 3), as a Gal transferase in presence of GalNAcβ-3Gal to form the SSEA-3 antigen (Galβ-3GalNAcβ-3Gal).

57. A method for producing an oligosaccharide comprising the motif GalNAcβ-3Gal, comprising culturing a microorganism which is galP (galactose permease), LacZ− (βgalactosidase), MelA− (α-galactosidase) and wbpP encoding for UDP-GlcNAc-C4 epimerase, such as the Pseudomonas aeruginosa wbpP gene; or a gne gene encoding for a UDP-glucose 4-epimerase, such as the gne gene of C. jejuni strain NCTC 11168 of SEQ ID No 9, in a culture medium comprising galactose, wherein said microorganism comprises a heterologous lgtD gene encoding α-1,4-Gal transferase which transfers a GalNAc residue to galactose to form the an oligosaccharide comprising GalNAcβ-3Gal.

58. The method of claim 57, wherein the lgtD gene allowed to further transfer a galactose moiety from UDP-Gal to GalNAcβ-3Gal to form the SSEA-3 antigen (Galβ-3GalNAcβ-3Gal).

59. The method of claim 58, which further comprises producing the terminal tetrasaccharide epitope of the SSEA-4 antigen (NeuAcα-3Galβ-3GalNAcβ-3Gal) and wherein the microorganism further comprises a heterologous gene encoding an α-3 sialylltranferase to transfer a sialic acid moiety from CMP-NeuAc to Galβ-3GalNAcβ-3Gal to form NeuAcα-3Galβ-3GalNAcβ-3Gal.

60. The method of claim 58, which further comprises producing the terminal tetrasaccharide epitope of the Globo-H antigen (Fucα-2Galβ-3GalNAcβ-3Gal) and wherein the microorganism further comprises a heterologous futC gene encoding an α-2 fucosyltranferase to transfer a fucose moiety from GDP-Fuc to Galβ-3GalNAcβ-3Gal to form Fucα-2Galβ-3GalNAcβ-3Gal.

61. A microorganism as defined in claim 58.

62. A microorganism as defined in claim 59.

63. A microorganism as defined in claim 60.

64. A culture medium comprising galactose and the microorganism of claim 61.
